(19) United States
(12) Patent Application Publication
(10) Pub. No.: US 2024/0245573 A1
(43) Pub. Date: Jul. 25, 2024

(54) DEVICE, METHOD, AND SYSTEM FOR PELVIC FLOOR REHABILITATION

(71) Applicant: Hyivy Health Inc., Kitchener (CA)

(72) Inventors: Rachel Bartholomew, Kitchener (CA); Kamyar Rouindej, Kitchener (CA)

(21) Appl. No.: 18/428,831

(22) Filed: Jan. 31, 2024

Related U.S. Application Data
(63) Continuation-in-part of application No. 17/549,538, filed on Dec. 13, 2021.
(60) Provisional application No. 63/124,595, filed on Dec. 11, 2020.

(51) Int. Cl. A61H 19/00; A61F 7/12; A61K 9/00; A61K 31/00; A61K 31/167; A61K 31/485; A61K 31/5513; A61K 31/573; A61M 31/00

(52) U.S. Cl. CPC A61H 19/40; A61F 7/12; A61K 9/0034; A61K 31/167; A61K 31/485; A61K 31/5513; A61K 31/573; A61K 31/658; A61M 31/00; A61H 2201/0153; A61H 2201/105; A61M 2205/123; A61M 2205/3303; A61M 2210/1475

(57) ABSTRACT

Provided herein are system, devices, and methods for a pelvic floor rehabilitation device. The rehabilitation device includes a handle for holding and manipulating the pelvic floor rehabilitation device, a controller to receive instructions and to control operation of the pelvic floor rehabilitation device, a shaft extending from the handle, wherein at least part of the shaft dilates based on input from the controller, at least one sensor for collecting data during operation of the pelvic floor rehabilitation device, and a lubrication system operable to introduce a substance into the vagina.

### TECHNICAL FIELD

The embodiments disclosed herein relate to pelvic floor rehabilitation, and, in particular to systems, methods, and devices for pelvic floor rehabilitation.

### INTRODUCTION

Pelvic floor rehabilitation refers to the treatment of various physiological complications affecting the vaginal canal, vaginal wall, or pelvic floor. These complications may often occur following pregnancy, vaginal birth and/or menopause, and may occur as a result of gynecological conditions such as endometriosis, pelvic organ prolapse, pelvic cancers and PCOS, vaginismus, or may occur after pelvic surgeries, and may include symptoms such as vaginal dryness, discomfort, inflammation and swelling, vaginal thinning, bleeding, rashes and irritation, muscle weakness and overactive response, discharge and infection, bowel and bladder incontinence, vaginal stenosis, vaginal atrophy, and pelvic pain. These symptoms and diagnosis directly impact quality of life, personal relationships, and day to day activities.

Currently a user faces several challenges if they need or want to apply pelvic floor rehabilitation therapy on their own. Previous systems, devices, and methods required a second individual, such as a healthcare professional, to assist a user in applying the pelvic floor rehabilitation therapy. In person exams and treatment rely on subjective, visual exams causing inconsistent treatment, condition management and diagnosis, highlighting the need for quantitative measurements for better characterization of pelvic health.

Current pelvic floor rehabilitation therapies do not implement data analysis on data collected during the pelvic floor rehabilitation in order to improve subsequent pelvic floor rehabilitation sessions. With a lack of follow up and a hands-off approach to condition management, patients will re-enter the health care system with reoccurring and continual health conditions.

Accordingly, there is a need for new pelvic floor rehabilitation devices, methods, and systems for use by people who have had pelvic cancers or pelvic diseases to help improve pelvic health after a devastating diagnosis, treatment, or surgery. Current pelvic floor rehabilitation devices, including static manual dilators and Kegel exercisers take an isolated approach to treating symptoms, focusing on one or two symptoms at most, have low adherence rates and have low improvement rates.

Further, systems and methods for the proper collection of data, both from the sensors in the device and the patient medical history, preferences and feedback are needed. A pelvic floor rehabilitation device and methods for data collections are provided.

### SUMMARY

Provided herein is a pelvic floor rehabilitation device including a handle for holding and manipulating the pelvic floor rehabilitation device, a controller to receive instructions and to control operation of the pelvic floor rehabilitation device, a shaft extending from the handle, wherein at least part of the shaft dilates based on input from the controller, at least one sensor for collecting data during operation of the pelvic floor rehabilitation device, and a lubrication system operable to introduce a substance into the vagina.

The substance introduced by the lubrication system may include at least a pharmaceutical.

The pharmaceutical may be contained within a lubrication cartridge. The lubrication cartridge may contain at least one of a lubricant and a fluid.

The at least a pharmaceutical may be personalized for a patient.

The pharmaceutical may be used for at least one of desensitization, pain relief, and hormone replacement.

The pharmaceutical may be at least one of an estrogen, a progesterone, lidocaine, morphine, valium, a muscle relaxant, cannabidiol (CBD), and tetrahydrocannabinol (THC).

The lubrication system may include an identification system for identifying the type of lubrication cartridge inserted into the lubrication system.

The identification system may include a barcode scanner to scan a barcode on the lubrication cartridge to identify the lubrication cartridge.

The lubrication system may include a puncturing mechanism for puncturing the lubrication cartridge when the lubrication cartridge is inserted into the lubrication system, wherein a puncture hole produced by the needle allows for drawing the contents from the lubrication cartridge by the lubrication system.

The pelvic floor rehabilitation device of claim 1 may further comprise a transmitter for transmitting data collected by the at least one sensor to at least a first computing device.

The pelvic floor rehabilitation device of claim 1 wherein the controller may be communicatively coupled to a computing device which transmits instructions for operation of the pelvic floor rehabilitation device.

The pelvic floor rehabilitation device of claim 1 wherein the handle may include at least one input control engageable by a user and communicatively coupled to the controller to control operation of the pelvic floor rehabilitation device.

The pelvic floor rehabilitation device may further comprise a temperature adjusting system operable to alter a temperature in a vagina.

The at least one sensor may comprise a pressure sensor to detect a resistance from a vagina.

The at least one sensor may comprise a force sensor to measure the force of dilation on a vagina.

The at least one sensor may comprise a temperature sensor to measure the temperature inside a vagina.

The at least one sensor may comprise a moisture sensor to measure a level of moisture inside a vagina.

Provided herein is a pelvic floor rehabilitation device including a handle for holding and manipulating the pelvic floor rehabilitation device, a controller to receive instructions and to control operation of the pelvic floor rehabilitation device, a shaft extending from the handle, wherein at least part of the shaft dilates based on input from the controller, at least one sensor for collecting data during operation of the pelvic floor rehabilitation device, and a temperature adjusting system operable to provide heating and cooling.

The temperature adjusting system may include at least one temperature adjusting element disposed on the shaft.

The at least one temperature adjusting element may comprise at least one thermoelectric module, wherein the at least one thermoelectric module provides heating and cooling. The thermoelectric module may be copper-based.

The at least one temperature adjusting element may comprise at least one thermoelectric module to provide cooling and at least one resistive heating element to provide heating.

The pelvic floor rehabilitation device may further comprise a lubrication system operable to introduce at least one pharmaceutical into the vagina, wherein the temperature adjusting system provides heating and cooling to control absorption of the at least one pharmaceutical.

The pelvic floor rehabilitation device may further comprise a transmitter for transmitting data collected by the at least one sensor to at least a first computing device.

The controller may be communicatively coupled to a computing device which transmits instructions for operation of the pelvic floor rehabilitation device.

The handle may include at least one input control engageable by a user and communicatively coupled to the controller to control operation of the pelvic floor rehabilitation device.

The at least one sensor may comprise a pressure sensor to detect a resistance from a vagina.

The at least one sensor may comprise a force sensor to measure the force of dilation on a vagina.

The at least one sensor may comprise a temperature sensor to measure the temperature inside a vagina.

The at least one sensor may comprise a moisture sensor to measure a level of moisture inside a vagina.

Provided herein is a remote therapeutic monitoring method for pelvic floor rehabilitation comprising receiving a treatment plan, by a patient, from a healthcare professional, wherein the treatment plan is for the operation and use of a pelvic floor rehabilitation device comprising a controller to receive instructions and to control operation of the pelvic floor rehabilitation device and at least one sensor for collecting data during operation of the pelvic floor rehabilitation device, connecting, from a remote location, to the pelvic floor rehabilitation device by a computing device of the healthcare professional, wherein the computing is communicatively coupled to the pelvic floor rehabilitation device and the at least one sensor, initiating, by the patient, a pelvic floor rehabilitation device treatment session according to the treatment plan, collecting sensor data from the at least one sensor of the pelvic floor rehabilitation device, transmitting the sensor data to the first computing device of the healthcare professional in real-time for the healthcare professional to monitor the treatment session and communicate with the patient, and providing feedback to the patient from the healthcare professional based on the sensor data.

The healthcare professional may view the sensor data on a graphical user interface (GUI) of the computing device of the healthcare professional.

The computing device of the healthcare professional may include at least one input device for receiving input from the healthcare professional.

The healthcare professional may receive patient-reported data for monitoring the treatment session and the feedback provided is also based on the patient-reported data.

The pelvic floor rehabilitation device may include a transmitter for transmitting the sensor data.

The sensor data may be sent directly to the computing device of the healthcare professional from the pelvic floor rehabilitation device.

The sensor data may be sent to a cloud server from the pelvic floor rehabilitation device and then to the computing device of the healthcare professional.

The computing device of the healthcare professional may be connected to the pelvic floor rehabilitation device and receives the sensor data by a Bluetooth™ connection.

The feedback may include the healthcare professional remotely controlling the operation of the pelvic floor rehabilitation device based on the sensor data.

Remotely controlling the operation of the pelvic floor rehabilitation device may further include at least one of adding steps to the current treatment session, removing steps from the current treatment session, increasing parameters of the current treatment session, decreasing parameters of the current treatment session, and stopping the current treatment session.

The remote therapeutic monitoring method may further comprise Analyzing the sensor data.

Analyzing the sensor data may further include comparing the sensor data to previous sensor data. The previous sensor data may be from the patient. The previous sensor data may be from at least one different patient.

Analyzing the sensor data may include generating baseline measurements for a treatment plan.

Analyzing the sensor data may include tracking progress of a treatment plan.

Analyzing the sensor data may include generating baseline measurements for diagnosis or for other treatments.

Providing feedback based on the sensor data may include providing a new treatment plan.

The use case of the method may be one of tissue healing, muscle control and response, function of organs near the vagina, treatment of metabolic conditions, and nervous system response.

Provided herein is a pelvic floor rehabilitation device for diagnosing and treating pelvic floor dysfunction, the device comprising a handle for holding and manipulating the pelvic floor rehabilitation device, a controller to receive instructions and to control operation of the pelvic floor rehabilitation device, a shaft extending from the handle, wherein at least part of the shaft dilates based on input from the controller, at least one sensor for collecting data during operation of the pelvic floor rehabilitation device, and an electromyography (EMG) system for tracking and measuring pelvic floor dysfunction using surface electrodes on a surface of the shaft.

The pelvic floor rehabilitation device may further comprise pressure sensors, wherein the EMG system and the pressure sensors provide high resolution vaginal manometry.

The pelvic floor device may further comprise a lubrication system operable to introduce a substance into the vagina. The substance introduced by the lubrication system may include at least one pharmaceutical, wherein the pharmaceutical is used for at least one of desensitization, pain relief, and hormone replacement.

The pelvic floor rehabilitation device may further comprise a temperature adjusting system operable to alter a temperature in a vagina to accomplish at least one of desensitization of tissues, increasing recovery, and increasing blood flow.

Provided herein is a pelvic floor rehabilitation device which monitors nervous system response, the device comprising a handle for holding and manipulating the pelvic floor rehabilitation device, a controller to receive instructions and to control operation of the pelvic floor rehabilitation device, a shaft extending from the handle, wherein at least part of the shaft dilates based on input from the controller, a plurality of sensors for collecting data during operation of the pelvic floor rehabilitation device, wherein the plurality of sensors includes at least one nervous system sensor.

The at least one nervous system sensor may include at least one of a blood oxygen sensor, a heart rate sensor, an oxygen saturation (SpO2) sensor, a pulse oximeter, a radiation sensor, a pH sensor, an accelerometer, a proximity sensor, and a force sensor.

The pelvic floor device may further comprise a lubrication system operable to introduce a substance into the vagina. The substance introduced by the lubrication system may include at least one pharmaceutical, wherein the pharmaceutical is used for at least one of desensitization, pain relief, and hormone replacement.

The pelvic floor rehabilitation device may further comprise a temperature adjusting system operable to alter a temperature in a vagina to accomplish at least one of desensitization of tissues, increasing recovery, and increasing blood flow.

Provided herein is a pelvic floor rehabilitation device which monitors changes to muscles, other tissues, and nerves, the device comprising a handle for holding and manipulating the pelvic floor rehabilitation device, a controller to receive instructions and to control operation of the pelvic floor rehabilitation device, a shaft extending from the handle, wherein at least part of the shaft dilates based on input from the controller, at least one sensor for collecting data during operation of the pelvic floor rehabilitation device, and an ultrasound system for elastography and imaging.

The pelvic floor device may further comprise a lubrication system operable to introduce a substance into the vagina. The substance introduced by the lubrication system may include at least one pharmaceutical, wherein the pharmaceutical is used for at least one of desensitization, pain relief, and hormone replacement.

The pelvic floor rehabilitation device may further comprise a temperature adjusting system operable to alter a temperature in a vagina to accomplish at least one of desensitization of tissues, increasing recovery, and increasing blood flow.

Provided herein is a pelvic floor rehabilitation device comprising a handle for holding and manipulating the pelvic floor rehabilitation device, a controller to receive instructions and to control operation of the pelvic floor rehabilitation device, a shaft extending from the handle, wherein at least part of the shaft dilates based on input from the controller, at least one sensor for collecting data during operation of the pelvic floor rehabilitation device, and a camera for providing topographical images of a vaginal vault, wherein the images are used as data for baseline measurements to inform a treatment protocol.

The pelvic floor device may further comprise a lubrication system operable to introduce a substance into the vagina. The substance introduced by the lubrication system may include at least one pharmaceutical, wherein the pharmaceutical is used for at least one of desensitization, pain relief, and hormone replacement.

The pelvic floor rehabilitation device may further comprise a temperature adjusting system operable to alter a temperature in a vagina to accomplish at least one of desensitization of tissues, increasing recovery, and increasing blood flow.

Provided herein is a pelvic floor rehabilitation device for diagnosing and treating sexual dysfunction, the device comprising a handle for holding and manipulating the pelvic floor rehabilitation device, a controller to receive instructions and to control operation of the pelvic floor rehabilitation device, a shaft extending from the handle, wherein at least part of the shaft dilates based on input from the controller, at least one sensor for collecting data during operation of the pelvic floor rehabilitation device, and a vibration system.

The pelvic floor device may further comprise a lubrication system operable to introduce a substance into the vagina.

### BRIEF DESCRIPTION OF THE DRAWINGS

The drawings included herewith are for illustrating various examples of articles, methods, and apparatuses of the present specification. In the drawings:

FIG. 1 is a block diagram illustrating a pelvic floor rehabilitation computing system, in accordance with an embodiment.

FIG. 2 is a simplified block diagram of components of a mobile device or portable electronic device.

FIG. 3 is a block diagram of a pelvic floor rehabilitation device, according to an embodiment.

FIG. 4 is a flow diagram of method of collecting data during pelvic floor rehabilitation therapy, according to an embodiment.

FIG. 5A is a perspective view of a simplified pelvic floor rehabilitation device with a partially transparent exterior, according to an embodiment.

FIG. 5B is a front view of a simplified pelvic floor rehabilitation device of FIG. 5A with a partially transparent exterior, according to an embodiment.

FIG. 5C is a side view of a simplified pelvic floor rehabilitation device of FIG. 5A with a partially transparent exterior, according to an embodiment.

FIG. 6 is an exploded view of a simplified pelvic floor rehabilitation device with a partially transparent exterior, according to an embodiment.

FIG. 7 is an exploded view of the dilation system in a shaft of a simplified pelvic floor rehabilitation device, according to an embodiment.

FIG. 8 is an exploded view of the temperature system in the simplified pelvic floor rehabilitation device, according to an embodiment.

FIG. 9A is a top down view of the temperature system in the simplified pelvic floor rehabilitation device, according to an embodiment.

FIG. 9B is a bottom up view of the temperature system of FIG. 9A in the simplified pelvic floor rehabilitation device, according to an embodiment.

FIG. 10 is an exploded view of the lubrication system in the simplified pelvic floor rehabilitation device, according to an embodiment.

FIG. 11 is a diagram of a system for collecting pelvic floor rehabilitation data, according to an embodiment.

FIG. 12 is a circuit diagram of the circuit board of a pelvic floor rehabilitation device, according to an embodiment.

FIG. 13 is a circuit board diagram of the dilation system of the shaft of FIG. 12, according to an embodiment.

FIG. 14 is a block diagram of an electronic system of a pelvic floor rehabilitation device.

FIG. 15a is a perspective view of a pelvic floor rehabilitation device according to an embodiment.

FIG. 15b is a perspective view of a pelvic floor rehabilitation device, according to an embodiment.

FIG. 15c is a perspective view of a pelvic floor rehabilitation device, according to an embodiment.

FIG. 15d is a perspective view of a pelvic floor rehabilitation device, according to an embodiment.

FIG. 16a is a transparent perspective view of a pelvic floor rehabilitation device including a dilation system, according to an embodiment.

FIG. 16b is a transparent perspective view of a pelvic floor rehabilitation device including a dilation system, according to an embodiment.

FIG. 17 is a transparent perspective view of a pelvic floor rehabilitation device including a lubrication system, according to an embodiment.

FIG. 18 is a flow diagram of a method of use of a pelvic floor rehabilitation device system, according to an embodiment.

FIG. 19 is a flow diagram of a method of remote therapeutic monitoring of use of a pelvic floor rehabilitation device, according to an embodiment.

FIG. 20 is a transparent perspective partial view of a pelvic floor rehabilitation device with a sensor board which includes multiple different sensors, according to an embodiment.

### DETAILED DESCRIPTION

Various apparatuses or processes will be described below to provide an example of each claimed embodiment. No embodiment described below limits any claimed embodiment and any claimed embodiment may cover processes or apparatuses that differ from those described below. The claimed embodiments are not limited to apparatuses or processes having all of the features of any one apparatus or process described below or to features common to multiple or all of the apparatuses described below.

One or more systems described herein may be implemented in computer programs executing on programmable computers, each comprising at least one processor, a data storage system (including volatile and non-volatile memory and/or storage elements), at least one input device, and at least one output device. For example, and without limitation, the programmable computer may be a programmable logic unit, a mainframe computer, server, and personal computer, cloud based program or system, laptop, personal data assistance, cellular telephone, smartphone, or tablet device.

Each program is preferably implemented in a high level procedural or object oriented programming and/or scripting language to communicate with a computer system. However, the programs may be implemented in assembly or machine language, if desired. In any case, the language may be a compiled or interpreted language. Each such computer program is preferably stored on a storage media or a device readable by a general or special purpose programmable computer for configuring and operating the computer when the storage media or device is read by the computer to perform the procedures described herein.

A description of an embodiment with several components in communication with each other does not imply that all such components are required. On the contrary a variety of optional components are described to illustrate the wide variety of possible embodiments of the present invention.

Further, although process steps, method steps, algorithms or the like may be described (in the disclosure and/or in the claims) in a sequential order, such processes, methods, and algorithms may be configured to work in alternate orders. In other words, any sequence or order of steps that may be described does not necessarily indicate a requirement that the steps be performed in that order. The steps of processes described herein may be performed in any order that is practical. Further, some steps may be performed simultaneously.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

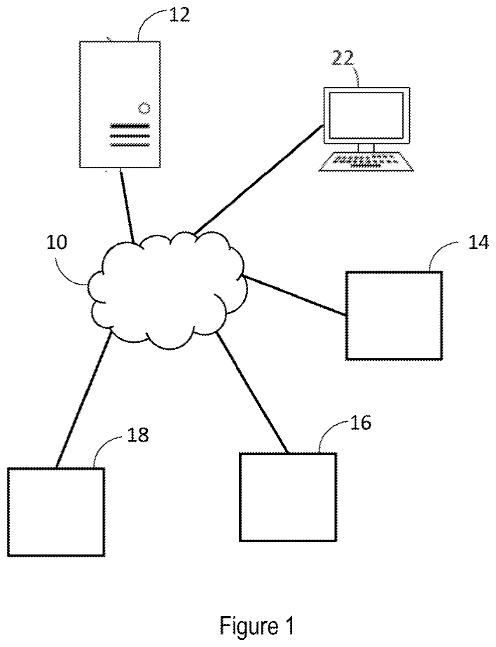

FIG. 1 shows a block diagram illustrating a pelvic floor rehabilitation computing system 10, in accordance with an embodiment.

The pelvic floor rehabilitation computing system 10 represents the various components of a system in which data is collected from at least one pelvic floor rehabilitation device and transferred to a computing device for analysis. The analysis may be done automatically, the user of the pelvic floor rehabilitation device may view their personal data on a personal device, and/or a healthcare professional may view the data on an interface. The pelvic floor rehabilitation computing system 10 is meant to show a high-level overview example of how data may move within a pelvic floor rehabilitation system and should not be taken as a specific example of how data moves between parties (e.g. the user and their associated healthcare professional(s)) or how the data is collected, stored, protected, etc., in an actual system.

The pelvic floor rehabilitation computing system 10 includes a plurality of pelvic floor rehabilitation devices 14, 16, 18. Each pelvic floor rehabilitation device 14, 16, 18 further includes at least one sensor configured to monitor a parameter during pelvic floor rehabilitation therapy. The pelvic floor rehabilitation devices 14, 16, 18 are configured to transmit data collected by the sensors. Three pelvic floor rehabilitation devices 14, 16, 18 are shown, however any number of pelvic floor rehabilitation devices may be possible in the pelvic floor rehabilitation system 10.

In the embodiment of FIG. 1, a system is represented whereby data can be collected from multiple devices and may include pooling of the data in order to perform of study of numerous patients each using a pelvic floor device wherein a healthcare professional or other researcher can determine the efficacy of the device for patients with a specific or general pelvic floor health concern. The data may not be pooled but may still go to a single healthcare professional who is treating multiple individual patients. In some embodiments the system may include just one patient and one healthcare professional.

The pelvic floor rehabilitation system 10 includes a server 12 configured to receive and store data transmitted by the plurality of pelvic floor rehabilitation devices 14, 16, 18. In some embodiments there may be no server and data may be stored locally on personal devices or on other computing devices.

The pelvic floor rehabilitation system includes a user device 22 configured to receive, store, and display the data collected by the pelvic floor rehabilitation devices 14, 16, 18. Although only one user device 22 is shown, any number of user devices may receive, store, and display the data. The user device 22 may be any device with a user interface capable of displaying the data, e.g. a mobile device or a personal computer. The user device 22 may also represent a device of a healthcare professional (or researcher) who is reviewing the data of their patient (or study participants).

The pelvic floor rehabilitation system 10 includes a server platform 12 which communicates with a plurality of pelvic floor rehabilitation devices 14, 16, 18 via a network 20. The server platform 12 also communicates with user device(s) 22. The server platform 12 may be a purpose built machine designed specifically for pelvic floor rehabilitation.

The devices 12, 14, 16, 18, 22 may include a connection with the network 20 such as a wired or wireless connection to the Internet. In some cases, the network 20 may include other types of computer or telecommunication networks. The devices 12, 14, 16, 18, 22 may include one or more of a memory, a secondary storage device, a processor, an input device, a display device, and an output device. Memory may include random access memory (RAM) or similar types of memory. Also, memory may store one or more applications for execution by processor. Applications may correspond with software modules comprising computer executable instructions to perform processing for the functions described below. Secondary storage devices may include a hard disk drive, floppy disk drive, CD drive, DVD drive, Blu-ray drive, or other types of non-volatile data storage. Processors may execute applications, computer readable instructions or programs. The applications, computer readable instructions or programs may be stored in memory or in secondary storage, or may be received from the Internet or other network 20. Input device may include any device for entering information into device 12, 14, 16, 18, 22. For example, input device may be a keyboard, keypad, cursor-control device, touch-screen, camera, or microphone. Display device may include any type of device for presenting visual information. For example, display device may be a computer monitor, a flat-screen display, a projector or a display panel. Output device may include any type of device for presenting a hard copy of information, such as a printer for example. Output devices may also include other types of output devices such as speakers, for example. In some cases, device 12, 14, 16, 18, 22 may include multiple of any one or more of processors, applications, software modules, second storage devices, network connections, input devices, output devices, and display devices.

Although devices 12, 14, 16, 18, 22 are described with various components, one skilled in the art will appreciate that the devices 12, 14, 16, 18, 22 may in some cases contain fewer, additional or different components. In addition, although aspects of an implementation of the devices 12, 14, 16, 18, 22 may be described as being stored in memory, one skilled in the art will appreciate that these aspects may also be stored on or read from other types of computer program products or computer-readable media, such as secondary storage devices, including hard disks, floppy disks, CDs, or DVDs; a carrier wave from the Internet or other network; or other forms of RAM or ROM. The computer-readable media may include instructions for controlling the devices 12, 14, 16, 18, 22 and/or processor to perform a particular method.

In the description that follows, devices such as server platform 12, pelvic floor rehabilitation devices 14, 16, 18 and user devices 22 are described performing certain acts. It will be appreciated that any one or more of these devices may perform an act automatically or in response to an interaction by a user of that device. That is, the user of the device may manipulate one or more input devices (e.g. a touchscreen, a mouse, or a button) causing the device to perform the described act. In many cases, this aspect may not be described below, but it will be understood.

As an example, it is described below that the devices 12, 14, 16, 18, 22 may send information to the server platform 12. For example, a user using the user device 22 may manipulate one or more input devices (e.g. a mouse and a keyboard) to interact with a user interface displayed on a display of the user device 22. Generally, the device may receive a user interface from the network 20 (e.g. in the form of a webpage). Alternatively or in addition, a user interface may be stored locally at a device (e.g. a cache of a webpage or a mobile application).

Server platform 12 may be configured to receive a plurality of information, from each of the plurality of pelvic floor rehabilitation devices 14, 16, 18 and user devices 22. Generally, the information may comprise at least an identifier identifying the pelvic floor rehabilitation device or user. For example, the information may comprise one or more of a username, e-mail address, password, or social media handle.

In response to receiving information, the server platform 12 may store the information in storage database. The storage may correspond with secondary storage of the device 12, 14, 16, 18, 22. Generally, the storage database may be any suitable storage device such as a hard disk drive, a solid state drive, a memory card, or a disk (e.g. CD, DVD, or Blu-ray etc.). Also, the storage database may be locally connected with server platform 12. In some cases, storage databases may be located remotely from server platform 12 and accessible to server platform 12 across a network for example. In some cases, storage databases may comprise one or more storage devices located at a networked cloud storage provider.

The plurality of pelvic floor rehabilitation devices 14, 16, 18 may be associated with a respective plurality of pelvic floor rehabilitation accounts. Similarly, the user device 22 may be associated with a user account. Any suitable mechanism for associating a device with an account is expressly contemplated. In some cases, a device may be associated with an account by sending credentials (e.g. a cookie, login, or password etc.) to the server platform 12. The server platform 12 may verify the credentials (e.g. determine that the received password matches a password associated with the account). If a device is associated with an account, the server platform 12 may consider further acts by that device to be associated with that account.

In the system of FIG. 1, any of the computing devices may include Bluetooth technology and connections between devices and transfer of data between devices my occur via Bluetooth.

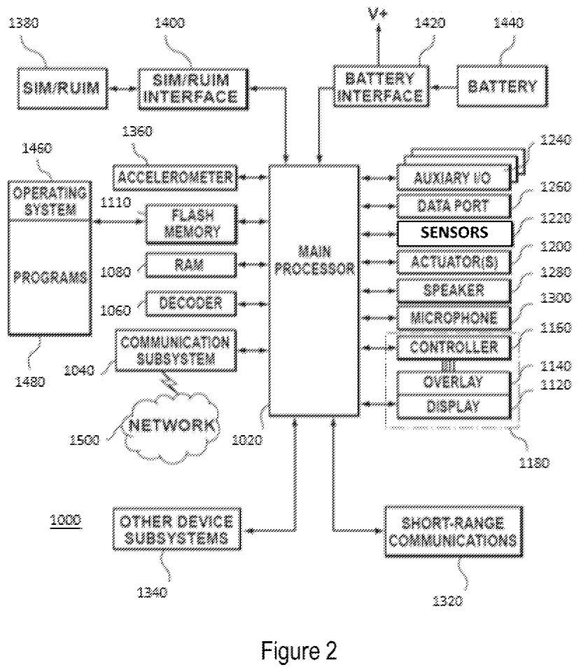

FIG. 2 shows a simplified block diagram of components of a mobile device or portable electronic device 1000. The portable electronic device 1000 includes multiple components such as a processor 1020 that controls the operations of the portable electronic device 1000. Communication functions, including data communications, voice communications, or both may be performed through a communication subsystem 1040. Data received by the portable electronic device 1000 may be decompressed and decrypted by a decoder 1060. The communication subsystem 1040 may receive messages from and send messages to a wireless network 1500.

The wireless network 1500 may be any type of wireless network, including, but not limited to, data-centric wireless networks, voice-centric wireless networks, and dual-mode networks that support both voice and data communications.

The portable electronic device 1000 may be a battery-powered device and as shown includes a battery interface 1420 for receiving one or more rechargeable batteries 1440.

The processor 1020 also interacts with additional subsystems such as a Random Access Memory (RAM) 1080, a flash memory 1100, a display 1120 (e.g. with a touch-sensitive overlay 1140 connected to an electronic controller 1160 that together comprise a touch-sensitive display 1180), an actuator assembly 1200, one or more sensors 1220, an auxiliary input/output (I/O) subsystem 1240, a data port 1260, a speaker 1280, a microphone 1300, short-range communications systems 1320 and other device subsystems 1340.

In some embodiments, user-interaction with the graphical user interface may be performed through the touch-sensitive overlay 1140. The processor 1020 may interact with the touch-sensitive overlay 1140 via the electronic controller 1160. Information, such as text, characters, symbols, images, icons, and other items that may be displayed or rendered on a portable electronic device generated by the processor 102 may be displayed on the touch-sensitive display 118.

To identify a subscriber for network access according to the present embodiment, the portable electronic device 1000 may use a Subscriber Identity Module or a Removable User Identity Module (SIM/RUIM) card 1380 inserted into a SIM/RUIM interface 1400 for communication with a network (such as the wireless network 1500). Alternatively, user identification information may be programmed into the flash memory 1100 or performed using other techniques.

The portable electronic device 1000 also includes an operating system 1460 and software components 1480 that are executed by the processor 1020 and which may be stored in a persistent data storage device such as the flash memory 1100. Additional applications may be loaded onto the portable electronic device 1000 through the wireless network 1500, the auxiliary I/O subsystem 1240, the data port 1260, the short-range communications subsystem 1320, or any other suitable device subsystem 1340.

In use, a received signal such as a text message, an e-mail message, web page download, or other data may be processed by the communication subsystem 1040 and input to the processor 1020. The processor 1020 then processes the received signal for output to the display 1120 or alternatively to the auxiliary I/O subsystem 1240. A subscriber may also compose data items, such as e-mail messages, for example, which may be transmitted over the wireless network 1500 through the communication subsystem 1040.

For voice communications, the overall operation of the portable electronic device 1000 may be similar. The speaker 1280 may output audible information converted from electrical signals, and the microphone 1300 may convert audible information into electrical signals for processing.

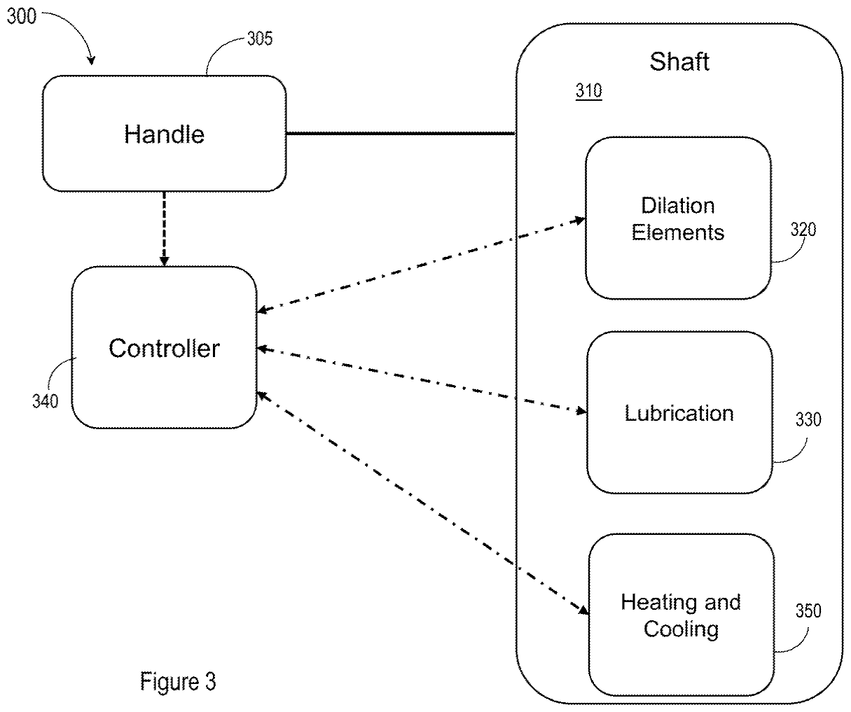

Referring to FIG. 3, illustrated therein is block diagram of a pelvic floor rehabilitation device 300 with a dilation system, a lubrication system 330, according to an embodiment. The pelvic floor rehabilitation device 300 includes a handle 305 for holding the pelvic floor rehabilitation device 300.

In some embodiments of a pelvic floor rehabilitation device there is no lubrication system.

The pelvic floor rehabilitation device 300 includes a controller 340 with input controls. In some embodiments, the controller is integrated with the handle (represented by dashed line between controller 340 and handle 305) and allows a user of the pelvic floor rehabilitation device 300 to view the input controls while operating the pelvic floor rehabilitation device 300 to provide individual pelvic floor rehabilitation therapy.

The pelvic floor rehabilitation device 300 includes a shaft 310 extending from the handle 305 (represented by solid line). The shaft includes dilation elements 320 which expand radially based on input from the controller 340 (dashed-dotted arrow representing communicative relationship between the components). The dilation elements are part of a dilation system which is held within the pelvic floor rehabilitation device. Components of the dilation system may be within the handle 305 as well as the shaft 310.

The pelvic floor rehabilitation device 300 includes a lubrication system 330 at least partially within the shaft 310, having a lubricant pump for releasing a lubricant 135 based on input from the controller 340 (dashed and dotted line). Various components of the lubrication system, for example the lubricant pump, may be disposed in the handle 305.

The pelvic floor rehabilitation device 300 includes a temperature system 350 at least partially within the shaft 310 to apply heating or cooling to the vaginal wall. The temperature system 350 adjusts temperature based on input from the controller 340 (dashed and dotted line). Various components of the temperature system may be disposed in the handle 305.

The pelvic floor rehabilitation device 300 includes at least one sensor for collecting data. The at least one sensor may be a pressure sensor to monitor the function of the dilation system (measuring dilation of the dilation elements of pressure from the vaginal wall), a temperature sensor (measuring the temperature of the temperature system of the vagina/vaginal wall), or a moisture sensor (measuring a moisture level within the vagina). The pelvic floor rehabilitation device 300 includes a wireless transmitter for transmitting data collected by the at least one sensor to a user device.

In other embodiments the at least one sensor may include at least one electromyography (EMG) sensor for tracking and measuring pelvic floor dysfunction using surface electrodes on a surface of the pelvic floor rehabilitation device which provide mechanical and functional feedback. EMG sensors may work in concert with pressure sensors or pressure transducers to conduct high resolution vaginal manometry to measure pelvic floor dysfunction. Together the EMG sensors and pressure sensors/transducers provide a clinical use measurement tool capable of mapping the pelvic floor including muscle, other tissues, and surrounding structures. The EMG sensors and pressure sensors/transducers allow for data to be collected which enables the patient and their healthcare professional(s) to monitor progress from the perspective of tissue and muscle changes from pre-treatment to post-treatment using the pelvic floor rehabilitation device.

In other embodiments, other sensors on or within the pelvic floor rehabilitation device 300 may include at least one of a blood oxygen sensor, a heart rate sensor, an oxygen saturation (SpO2) sensor, a pulse oximeter, a radiation sensor, a pH sensor, an accelerometer, a proximity sensor, and a force sensor. The pelvic floor rehabilitation device may be used in combination with heart rate variability (HRV). These additional sensors provide allow for data to be collected which enables the patient and their healthcare professional(s) to understand the effect of the pelvic floor rehabilitation device on various aspects of the nervous system pre-treatment, during treatment, and post treatment. The blood oxygen sensor, heart rate sensor, oxygen saturation sensor, and pulse oximeter provide information about the nervous system via the cardiovascular system during treatment, including the effects of treatment on blood flow to the vaginal canal and the effects of changing temperature on heart rate and blood oxygen. The heart rate sensor and HRV also provide information on nerves and nervous system changes. The radiation sensor provides information on radiation stenosis changes. The pH sensor provides information on the effect of the introduction of lubrication or pharmaceuticals to the vagina. The accelerometer provides information on the effect of treatment on the strength of the pelvic floor muscles.

The lubrication system 330 of the pelvic floor rehabilitation device 300 is configured to accept a lubrication cartridge. In other embodiments, the lubrication system may employ means other than a cartridge to provide lubrication to the vagina. The lubrication cartridge may contain lubricant which is released by the lubrication pump 332. The lubricant may include pharmaceuticals for desensitization, pain relief, or therapeutic reasons (e.g. hormone replacement) as many patients experience vaginal dryness and pain, before treatment, during treatment, and after treatment For example, the pharmaceutical(s) may include an estrogen, a progesterone, other hormones, lidocaine, morphine, valium, other painkillers, muscle relaxants, cannabinol (CBD), and tetrahydrocannabinol (THC). The lubrication system may release only pharmaceuticals and not lubricant. The lubrication cartridges may contain varying amounts of lubricant, fluid, or pharmaceuticals to allow for a patient personalized therapy. That is, the number, dosage, and concentration of the various components introduced into the vagina by the lubrication system may be individually determined/prescribed for each patient. Each patient may have different formulations of components for different circumstances of use or as a therapy plan progresses.

Use of lubrication and/or pharmaceutical may be in combination with heating and/or cooling. The specific requirements will depend on the patient and may change during a treatment. The absorption of pharmaceuticals can be affected by the use of heating and/or cooling. The dryness/wetness of the vagina can be monitored and adjusted to prevent tearing due to dryness or infections/pH imbalance due to wetness.

In some embodiments, the pelvic floor rehabilitation device 300 may include an ultrasound system for elastography and imaging to map muscles, other tissues, and nerve locations. The ultrasound system can be used to monitor changes to muscles, other tissues, and nerves by collecting data before, during, and after treatment. This data can inform the patient and their healthcare professional(s) of progress that is being made through treatment.

In some embodiments the pelvic floor rehabilitation device 300 may include a camera to take photographs within the vaginal canal and to provide topographical images of the vaginal vault. The photographs provide data for baseline measurements to inform a treatment protocol and to track progress when the treatment protocol is applied. For example, the photographs may show the effects of dilation therapy, with or without heating/cooling and lubrication/pharmaceuticals, on stenosis or atrophy of the vagina.

In some embodiments the pelvic floor rehabilitation device 300 may include a vibration subsystem to cause vibration of the device 300 for therapeutic purposes and/or for sexual stimulation. That is, the patient may use the vibration system as part of treatment and/or may use the vibration system to monitor the effects of treatment (e.g., dilation, heating/cooling, and/or lubrication/pharmaceuticals) on their sexual health.

The pelvic floor rehabilitation device 300 may also include pleasure attachments.

In some embodiments the lubrication system includes an identification system for identifying the type of lubrication cartridge that is accepted. The identification system may include a barcode scanner to scan a barcode on the lubrication cartridge to identify the lubrication cartridge and its contents. The lubrication system may include a needle for puncturing the lubrication cartridge when accepting the lubrication cartridge. The puncture hole produced by the needle allows for drawing the contents from the lubrication cartridge by the lubrication system. The puncture may be made with any puncturing mechanism and is not limited to a needle.

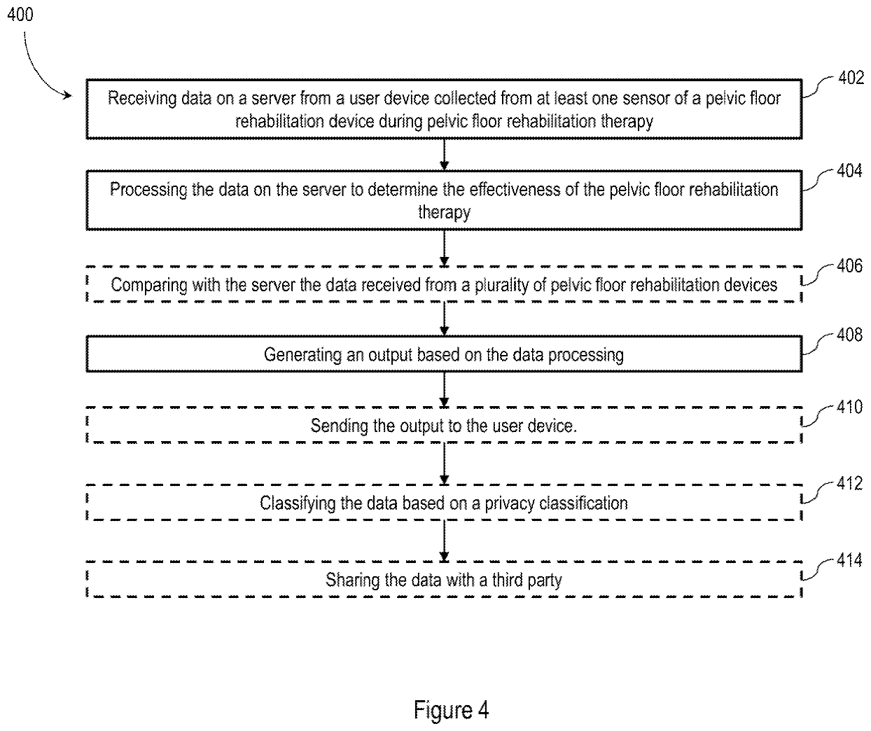

Referring to FIG. 4, illustrated herein is a flow diagram of method 400 of collecting data during pelvic floor rehabilitation therapy, according to an embodiment. The method 400 of collecting data during pelvic floor rehabilitation therapy includes receiving data on a server from a user device, at 402. The data is collected from at least one sensor of a pelvic floor rehabilitation device during pelvic floor rehabilitation therapy.

During a session of use, the user of the pelvic floor rehabilitation device starts a preprogrammed session in which various parameters are altered and controlled (e.g., a level of dilation of a dilation system, a temperature of a heating and/or cooling system, or a prescribed lubrication amount or type of a lubrication system). While the session is ongoing data is collected from at least one sensor as well as documenting the changes in the various parameters. The user may adjust the parameters as the session is occurring. The user cannot go beyond the preprogrammed parameters of the session.

The method 400 of collecting data during pelvic floor rehabilitation therapy also includes processing the data on the server to determine the effectiveness of the pelvic floor rehabilitation therapy, at 404.

The method 400 of collecting data during pelvic floor rehabilitation therapy includes optionally comparing with the server the data received from a plurality of pelvic floor rehabilitation devices, at 406.

The method 400 of collecting data during pelvic floor rehabilitation therapy includes generating an output based on the data processing, at 408.

The method 400 of collecting data during pelvic floor rehabilitation therapy includes optionally sending the output to the user device, at 410.

The method 400 of collecting data during pelvic floor rehabilitation therapy includes optionally classifying the data based on a privacy classification, at 412.

The method 400 of collecting data during pelvic floor rehabilitation therapy includes optionally sharing the data with a third party, at 414.

In some embodiments, the output is a time series of the data collected from the at least one sensor of a vaginal dilation device and displays the time series on the user device.

In some embodiments, the output is a pelvic floor rehabilitation therapy plan based on the data from the at least one sensor. The at least one sensor may be a pressure sensor, a force sensor, a moisture sensor, a temperature sensor, or a pH sensor. Any combination of sensors and any sensor relevant to pelvic floor rehabilitation is contemplated.

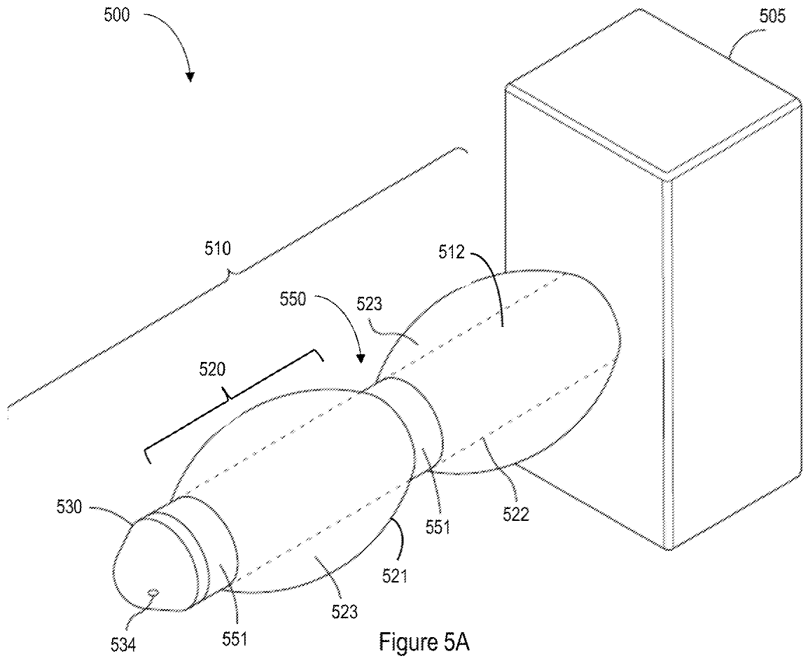

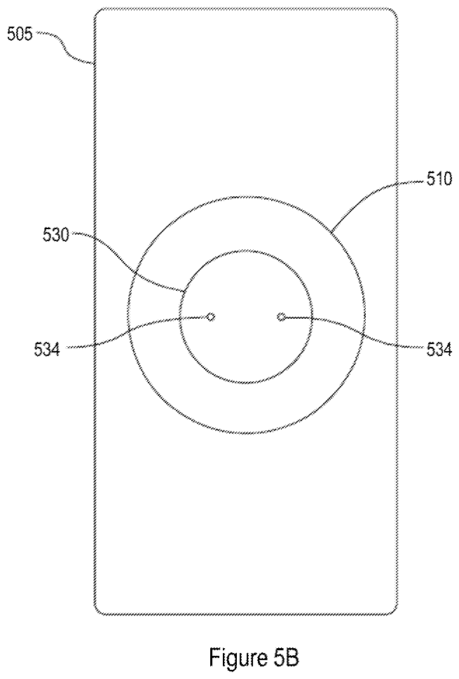

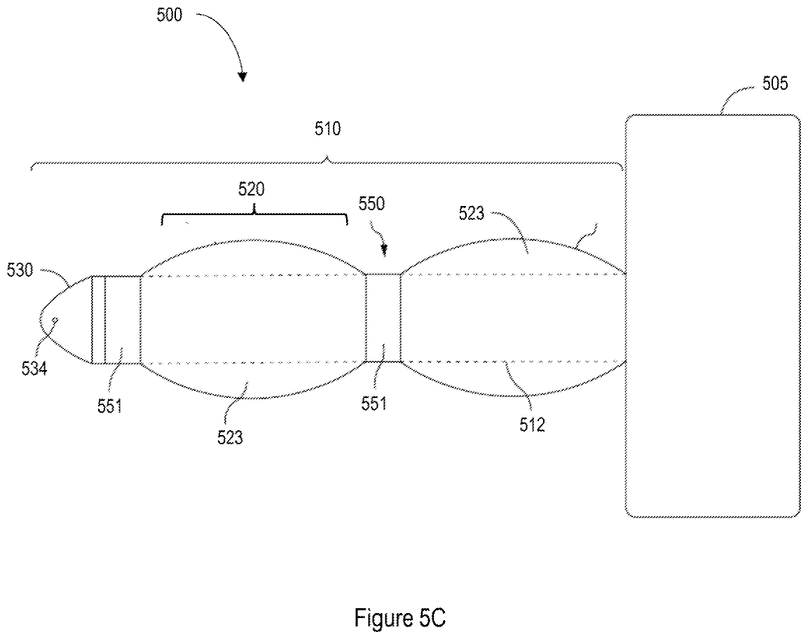

The pelvic floor rehabilitation device (and associated components), as shown in FIGS. 5A-10 is a simplified and exemplary embodiment which shows the functional capabilities of the pelvic floor rehabilitation device and the various systems within the pelvic floor rehabilitation device but is not intended to represent an actual design embodiment of a pelvic floor rehabilitation device.

Referring to FIG. 5A, illustrated herein is a perspective view of a pelvic floor rehabilitation device 500, according to an embodiment. The pelvic floor rehabilitation device 500 includes a handle 505 for holding the pelvic floor rehabilitation device 500.

In some embodiments the handle may be modular and may connect to a variety of sub-systems to provide differing functions. For example, a modular handle may be connected to a temperature system for providing heating and cooling to the pelvic floor. In other embodiments the handle may be connected to a lubrication system for releasing lubricant into the vaginal canal. In FIG. 5A the handle is connected to a shaft 510 which extends from the handle 505 and includes a dilation system.

The shaft 510 radially expands at two dilation elements 520 during pelvic floor rehabilitation (only one dilation segment called out to reduce clutter). The dilation elements 520 include an outer surface 521 which in use will contact the vaginal walls of the user and an inner surface 522 which is either reversibly or irreversibly attached to the shaft 510. The dilation elements are preferably permanently affixed to the shaft 510. The dilation elements may comprise silicone and may be over molded onto the outer shell of the shaft 510. Between the outer surface 521 and the inner surface 522 is an interior 523 which is inflated to expand the dilation elements. The inner surface of the dilations elements 520 encompasses and is in contact with an inner tube 512 of the shaft. The inner surface 522 may comprise a plastic that does not inflate or change shape. The outer surface 521 of the dilations elements is inflatable. The outer surface 521 of the dilation elements 520 is preferably made of silicone, but may be made of an elastic polymer, or any other suitable material. In the embodiment of FIG. 5A there are two dilation elements but in other embodiments any number of dilation elements may be employed.

In some embodiments, the radial expansion of the dilation elements 520 may be calibrated individually for each user. The calibration may include, without limitation, level of expansion, rate of expansion, and differential expansion between dilation elements may be calibrated for each user.

The shaft may include a pressure sensor. The pressure sensor may be inside the dilation element 520 within interior 523 for sensing the atmospheric pressure within the dilation element. Air may be pumped into the interior 523 and the air pressure required for radial expansion may be measured. The pressure difference between expansion of the dilation element outside the vaginal canal and inside the vaginal canal may be used to calculate the vaginal resistance for dilation. In other embodiments the pressure sensor may be capable of measuring both the air pressure within the dilation elements as well as the force applied to the pressure sensor by the vaginal wall. In other embodiments two separate sensors may be used to measure the air pressure within the dilation elements and the pressure from the vaginal wall. In some embodiments, the vaginal resistance may be calculated using a pressure sensor alone, a force sensor alone, or a pressure sensor and force sensor in combination. The vaginal resistance may be used to adjustably add more power to the motor for pumping air into the dilation element 520. In some embodiments, the vaginal resistance may be used to progressively reduce the dilation of the dilation element 520 after an initial overcompensating dilation.

The pressure sensor data may be used to approximate the diameter of the dilation element to correlate the diameter with a prescribed diameter for the user's session. The pressure sensor data may also be used to ensure safety during operation of the dilation system such that the dilation elements do not go beyond a prescribed amount of dilation.

Data collected from the various pressure and/or force sensors may be sent to a computing device via a microcontroller within the pelvic floor rehabilitation device which is communicatively coupled to the sensor(s). The microcontroller of the pelvic floor rehabilitation device is discussed in greater detail below.

The pump used for pumping air into the interior 523 of the dilation element 520 may be any type of pump capable of pumping air. The pump includes, without limitation, electronic and hand pumps. The pump may modulate the pressure inside the interior 523 to allow for variable expansion of the shaft 510. The pressure, rate of expansion, and amount of expansion may be modulated to allow for a low pressure massage for providing elasticity to the surrounding tissues, or a high pressure massage for providing medical deep tissue therapy.

In some embodiments, the dilation elements 520 may be made out of any material with sufficient elasticity to withstand the stretching of the dilation element 520. The dilation element material includes, but is not limited to, medical grade silicone or rubber.

The pelvic floor rehabilitation device 500 also includes a lubrication system 530 connected to the shaft 510, having a lubricant pump for releasing a lubricant. The shaft 510 includes the inner tube 512 whereby the lubricant may flow through. The lubricant is released through two openings 534 at the distal end of the lubrication system 530.

In some embodiments, the lubrication system 530 includes a moisture sensor for detecting moisture in a vaginal canal. The lubrication system may be configured to release lubricant when the moisture sensor detects low moisture levels inside the vaginal canal. The moisture sensor may be, without limitation, a humidity sensor, a resistive moisture sensor, capacitive soil moisture sensor, or any combination therein. The data from the moisture sensor may be sent to a microcontroller as discussed below.

In some embodiments, the lubrication system includes a flow sensor to monitor the release of lubricant by the lubrication system 530. Based on the flow sensor measurements the lubrication system 530 may modulate the release of lubricant by the lubricant pump to allow for a constant flow of lubricant in the vaginal canal. The lubrication system includes a one-way valve such that the lubricant only released outwards from the pelvic floor rehabilitation device 500.

In some embodiments, the pelvic floor rehabilitation device connects with a user device, such as a smartphone, running an application which is able to track a user's medication usage. The mobile application may also provide the user reminders to take their medication at timed intervals.

In some embodiments, the lubrication system includes a self-cleaning function which allows for cleaning of the lubrication system after it is used.

Referring to FIG. 5B, illustrated herein is a front view of pelvic floor rehabilitation device 500, according to an embodiment. The distal end of the lubrication system 530 includes two openings 534 for release of the lubricant. The distal end of the lubrication system 530 may be narrower than the shaft 510 to allow for easier penetration into the vaginal canal. In some embodiments, the distal end of the lubrication system 530 may be wider than the shaft 510 to act as an anchor during use.

In some embodiments the shaft may include a pH sensor for determining a pH level of the vaginal fluid. In some embodiments the shaft may include a photoplethysmogram (PPG) or more specifically a vaginal PPG (VPP) which can measure blood volume change within blood vessels of tissues of the body and detect arousal in the user.

Referring to FIG. 5C, illustrated herein is a side view of a pelvic floor rehabilitation device 500 with a partially transparent exterior, according to an embodiment. The pelvic floor rehabilitation device includes a temperature system 550 to generate heat and/or generate cooling. The temperature system may adjust the temperature of the shaft 510. The temperature system 550 includes conductive rings 551 along the shaft and a heat exchanger to modulate the temperature of the conductive rings 551. In other embodiments, heating and cooling may be achieved by other means and may be part of two different systems. The different systems may be modular.

In some embodiments, the temperature system 550 adjusts the temperature of the lubricant released by the lubrication system 530. The temperature system 550 may also adjust the temperature of the lubrication cartridge. In some embodiments, the temperature system 550 adjusts the temperature inside the vaginal canal by adjusting the temperature of at least part of the shaft 510.

In some embodiments, the pelvic floor rehabilitation device 500 includes a temperature sensor to measure the temperature inside a vaginal canal. The temperature system 550 may use the measurements from the temperature sensor to adjust the amount of heating or cooling provided to at least part of the pelvic floor rehabilitation device 500 or the lubricant. In some embodiments the temperature system 550 automatically adjusts the amount of heating or cooling based on the temperature sensor or a preprogrammed plan. In some embodiments, a user or a medical provider may manually adjust the amount of heating or cooling while providing pelvic floor rehabilitation therapy.

The temperature sensor measurements may be used to increase the heat generated by the temperature system 550 when the vaginal canal temperature is below a threshold temperature.

The temperature sensor measurements may be used to increase the cooling generated by the temperature system 550 when the vaginal canal temperature is above a threshold temperature.

The temperature sensor measurements may be used to determine a preferred vaginal canal temperature for pelvic floor rehabilitation therapy based on the temperature measurements collected from a plurality of pelvic floor rehabilitation devices 500.

Data from the temperature sensor may be used as a safety control to ensure that the temperature does not surpass or go below safety limits or beyond what has been designated in the preprogrammed session plan.

Data from the temperature sensor may be sent to a microcontroller as discussed below.

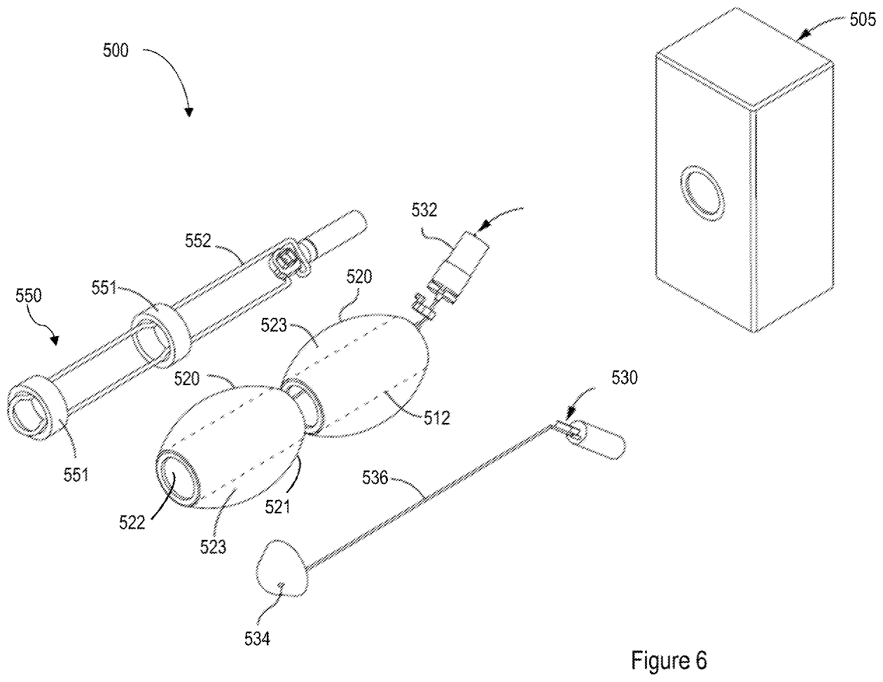

Referring to FIG. 6, illustrated herein is an exploded view of pelvic floor rehabilitation device 500, according to an embodiment. The pelvic floor rehabilitation device 500 includes a temperature system for generating heating and cooling. The temperature system includes temperature adjusting elements 551 which allow for transfer of heating and/or cooling to the vaginal canal. In some embodiments the exterior of the device 500 may be, without limitation, fully transparent, partially transparent, or opaque.

The same temperature system may be used for both heating and cooling without requiring any parts of the pelvic floor rehabilitation device to be switched out. The use of a single temperature system for both heating and cooling increases usability for the patient/user as they are not required to switch out parts. The single temperature system also allows for more rapid changes in temperature during a single treatment period instead of requiring the patient/user to stop and start to switch out parts. The single temperature system also minimizes the chance of injury to the patient as it eliminates the need for removable parts, openings, etc., which would be required if parts needed to be switched out.

In the embodiment of FIG. 6 the temperature adjusting elements are conductive rings. In other embodiments the temperature adjusting elements may be resistive heating elements. In other embodiments the temperature adjusting elements may be thermoelectric or “Peltier” modules which enable both heating and cooling. Cooling enables desensitization and recovery for tissues while heating increase blood flow. In some embodiments the temperature adjusting elements may include both resistive heating elements and thermoelectric modules wherein the resistive heating elements provide heating and the thermoelectric modules provide cooling. In embodiments with thermoelectric modules, the thermoelectric modules may be copper-based to achieve rapid cooling.

In an embodiment of the pelvic floor rehabilitation device which includes a lubrication system, the adjustment of temperature by the temperature system may provide a mechanism to control absorption of pharmaceuticals through heating and cooling. Controlling absorption ensures that less pharmaceuticals are wasted and that the proper does is received by the patient which enhances the therapeutic experience.

Between the temperature adjusting elements 551 are the dilation elements 520 which radially expand with air pumped by the air pump. An inner tube 512 of the shaft allows for the elements of the temperature system 550, various sensors (not shown), and lubrication system 530 to run through the shaft 510. A lubrication delivery tube 536 runs through the inner tube 512 for release of lubricant by the lubrication system 530.

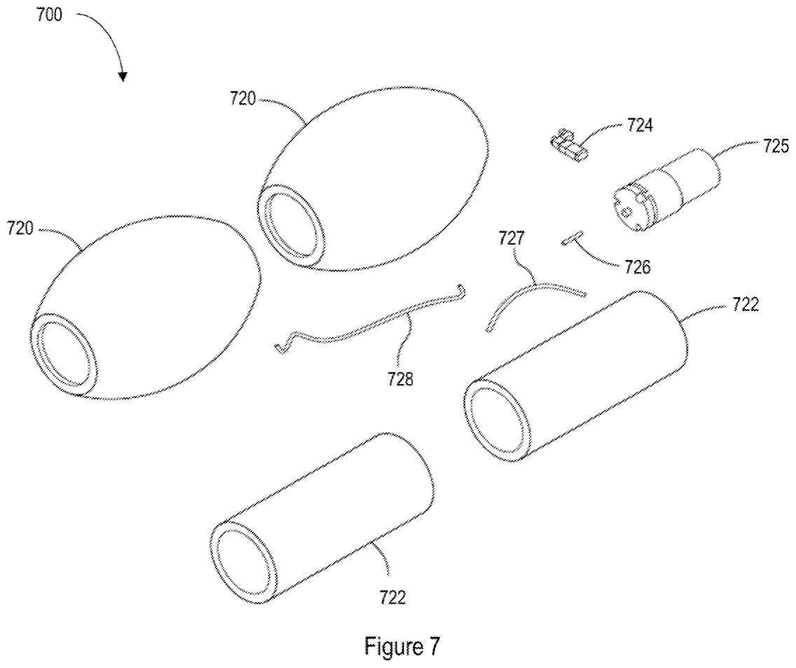

Referring to FIG. 7, illustrated herein is an exploded view of a dilation system 700 in a shaft of a pelvic floor rehabilitation device, according to an embodiment. The dilation system is a pneumatic dilation system that performs the physical dilation of the vaginal canal using a pneumatic mechanism. The pneumatic dilation system inflates in a radial direction at two different dilation elements 720 controlled separately by a controller.

The dilation is achieved by pneumatic inflation of the dilation elements 720. Manipulation of two different dilation elements 720 is done by the direction control valve 724. A controller of the pelvic floor rehabilitation device controls the direction control valve 724.

An outer surface of the dilation element 720 is made of silicone that expands to provide dilation when pressurized. The wall inflates like a balloon radially.

A direction control valve 724 controls the flow of pressure that has been created by air pump 725 to the dilation element. An inner surface of the dilation element 720 includes a connector for connecting tubing from the direction control valve 724 to an interior of the dilation element 720. The connector may be a right-angled tubing connector. The air pump 725 may inflate both the dilation elements 720 individually as well as together at the same time. The air pump 725 may also inflate the dilation elements 720 in sequence. The direction control valve 724 may also control deflation of the dilation elements 720.

The direction control valve 724 may be a solenoid valve.

The air pump 725 is controlled by a microcontroller as discussed below.

In some embodiments, a solid inner surface 722 of the dilation element 720 is made of plastic that does not inflate or change shape when pressurized. The solid inner surface 722 has pressure inlets coming from a first pneumatic hose 726 connected to the direction control valve 724.

The electric air pump 725 is controlled by a controller of the pelvic floor rehabilitation device. The air pump 725 generates pneumatic pressure in the hosing system necessary for the dilation procedure. The amount of pressure to be created to have different levels of dilation is achieved by time manipulated actuation of the air pump 725.

The first pneumatic hose 726 connects pump outlet to the hosing of the pneumatic dilation system. The second hose 727 provides pressure to the second dilation silicone catheter. The third hose 728 provides pressure to the first dilation silicone catheter. In some embodiments a single hose may be used for providing pressure to the dilation silicone catheter. In some embodiments, multiple hoses may be used for each dilation silicone catheter.

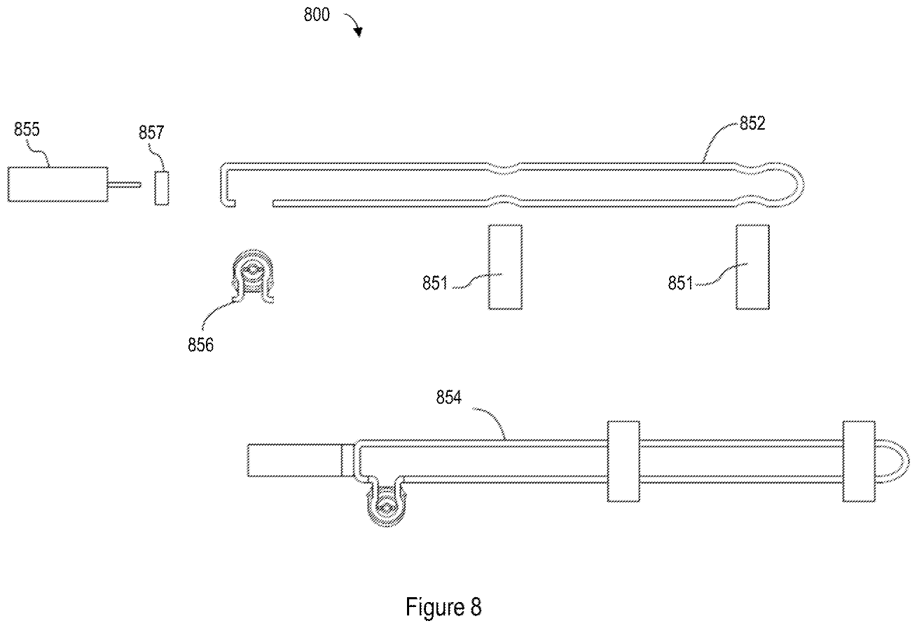

Referring to FIG. 8, illustrated herein is an exploded view of a possible temperature system 800 in the pelvic floor rehabilitation device, according to an embodiment. The temperature system 800 includes two temperature adjusting elements 851 which are placed in between the dilation elements. In FIG. 8 the temperature adjusting elements 851 are conductive rings 851 and the rest of temperature system shown is designed to work with conductive rings. In other embodiments, the temperature system may comprise resistive heating elements and the rest of the temperature system would be changed accordingly (i.e. electrical components to provide a current to the resistive heating elements instead of tubing to pump a fluid to conductive rings). The conductive rings 851 are connected via a tubing 852 which allows for transferring of heating or cooling to the conductive rings 851. A fluid is pumped through the tubing 852 of the temperature system 800 to the conductive rings 851 where the conductive rings 851 then conduct heating or cooling to the vaginal wall. The fluid is stored in a cartridge 853.

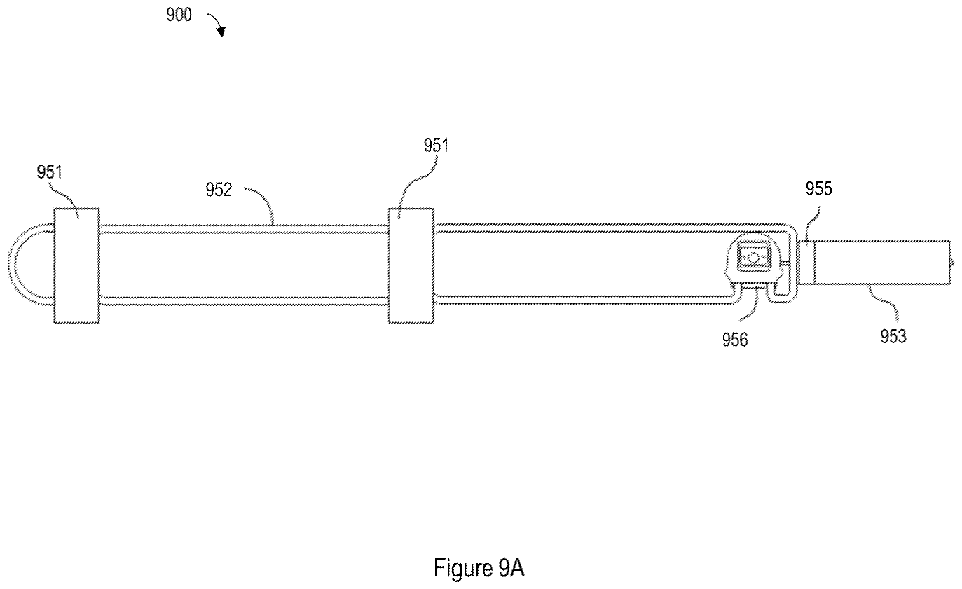

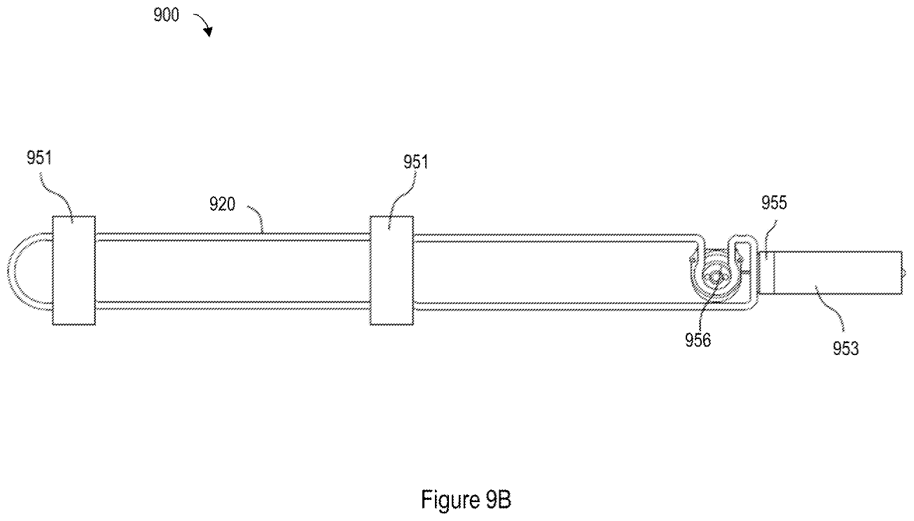

The temperature system 800 includes an external shell 854 which houses a heat exchanger 855 connected to a fluid pump 856. The fluid pump 856 transfers the fluid through tubing 852 to adjust the temperature of conductive rings 851. A surface 557 of the heat exchanger 855 where the cartridge 853 is placed rests beside the fluid pump 856 such that it is in contact with the tubing 852 in order to heat the fluid within the tubing 852. The fluid pump 856, found in the base of the pelvic floor rehabilitation device, is connected to the tubing 852 running through the core of the device, and to a controller responsible for controlling the fluid velocity. The tubing 852 is in contact with the conductive rings 851, which are a component of the hot and cold system shell. The shell is composed of two cylinders of plastic connected by conductive rings, as shown in the FIG. 9A below.

Referring to FIG. 9A, illustrated herein is a top down view of the temperature system 900 in the pelvic floor rehabilitation device, according to an embodiment. The temperature system 900 includes two conductive rings 951 which are connected via a tubing 952 which allows for transferring of heating or cooling to the conductive rings 951. The tubing 952 contains a fluid received from the cartridge 953 which is either heated or cooled by the heat exchanger 955. The fluid pump 956 pumps the fluid throughout the tubing 952 to transfer the heating or cooling to the conductive rings 951.

The temperature system 900 allows for administration of a hot and cold therapy which reduces pain and inflammation for people with pelvic health conditions. Cooling is responsible for reduction in inflammation, pain, desensitization, and discomfort, whereas heating is responsible for relaxation, increasing blood flow to promote tissue healing, increase in natural lubrication and reduced muscle tension within the pelvic area. Alternating between hot therapy and cold therapy sessions allows for a more effective lessening of pelvic symptoms and a quicker recovery from therapy.

Referring to FIG. 9B, illustrated herein is a bottom up view of the temperature system 900 in the pelvic floor rehabilitation device, according to an embodiment. The tubing 952 curves around the bottom of the pump 956 before connecting with the heat exchanger 955.

The temperature system 900 allows for the user to alternate between heat and cooling through insertion of a cartridge 953. The cartridge 953 may be either a hot cartridge or a cold cartridge to provide heating or cooling respectively to the heat exchanger 955. In some embodiments, the cartridge 953 is both a heating and cooling cartridge for providing both heating and cooling. The cartridge 953 is inserted into the pelvic floor rehabilitation device by the user and the heat or cold from the cartridge 953 is exchanged between the cartridge 953 and the fluid in the reservoir via a heat exchanger 955. The heat exchanger 955 is made of conductive materials to allow for rapid cooling or heating. The heated or cooled fluid is then pumped up and down the entire device in a closed loop tubing system. Heat exchange then occurs between the tubing and the conductive rings, which are on the device external, thus heating or cooling the pelvic area.

In some embodiments, the cartridge 953 may be inserted into the device at a cartridge compartment. In some embodiments, the cartridge compartment for the temperature system may be the same as a compartment for accepting a lubrication cartridge. In some embodiments the cartridge compartment for the temperature system is different from a compartment for accepting a lubrication cartridge. The cartridge compartment may be a component of the handle. In some embodiments, the handle with the cartridge compartment for the temperature system is removable and may be modularly replaced with a handle with the cartridge compartment for the lubrication system.

The temperature system is the middle subsystem of the pelvic floor rehabilitation device. The lubrication system is found internal to the temperature system, and the dilation system lies on the external of the shell.

In another embodiment of a heating system, resistive heating bands may be present on the shaft of the pelvic floor rehabilitation device. The resistive heating bands may heat up or cool down under the control of a microcontroller of the pelvic floor rehabilitation device. The resistive heating bands may include temperature sensors. The resistive heating bands may be mounted on the shaft with adhesive.

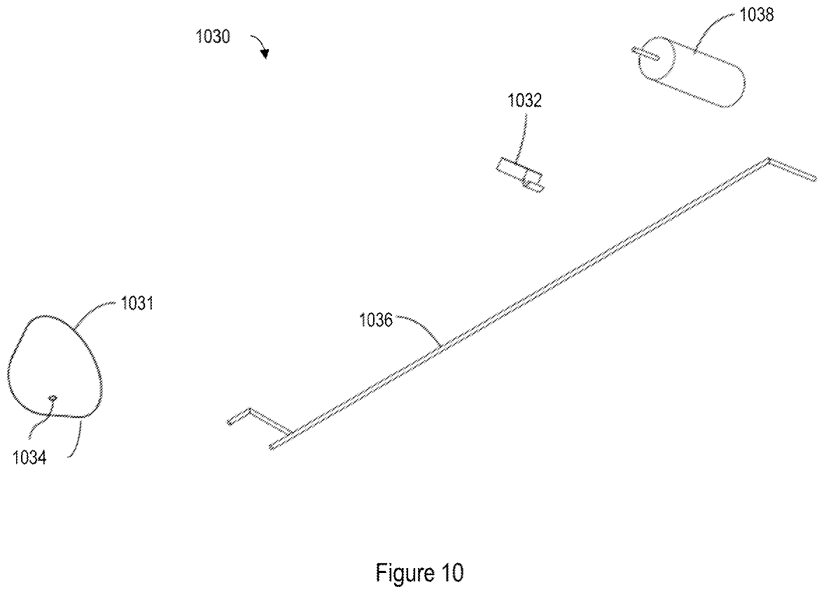

Referring to FIG. 10, illustrated herein is an exploded view of the lubrication system 1030 in a pelvic floor rehabilitation device, according to an embodiment. The lubrication system keeps the vagina lubricated in order to ease the insertion and placement of the device and as a result it improves patient comfort. The lubrication system includes a lubricant pump 1032 for releasing a lubricant based on input from a controller.

The lubrication system 1030 includes a lubrication cartridge 1038. The lubrication cartridge 1038 is a disposable canister which contains lubricant. The lubricant is preferably a medical grade lubricant, but may any suitable for use in a vaginal canal. The lubricant may also include a pharmaceutical.

The lubrication system 1030 includes tubing 1036 which delivers the lubricant from the lubrication cartridge 1038 to a tip 1031 of the lubrication system 1030 and releases it through an opening 1034 in the tip 1031 at a specific rate. The rate of lubrication release is based on the rate of release of lubricant from the lubricant pump 1032 into the tubing 1036.

The lubricant pump 1032 of the lubrication system 1030 is connected to a controller. The controller is able to control the rate of lubricant release and an on/off state of the lubricant pump 1032.

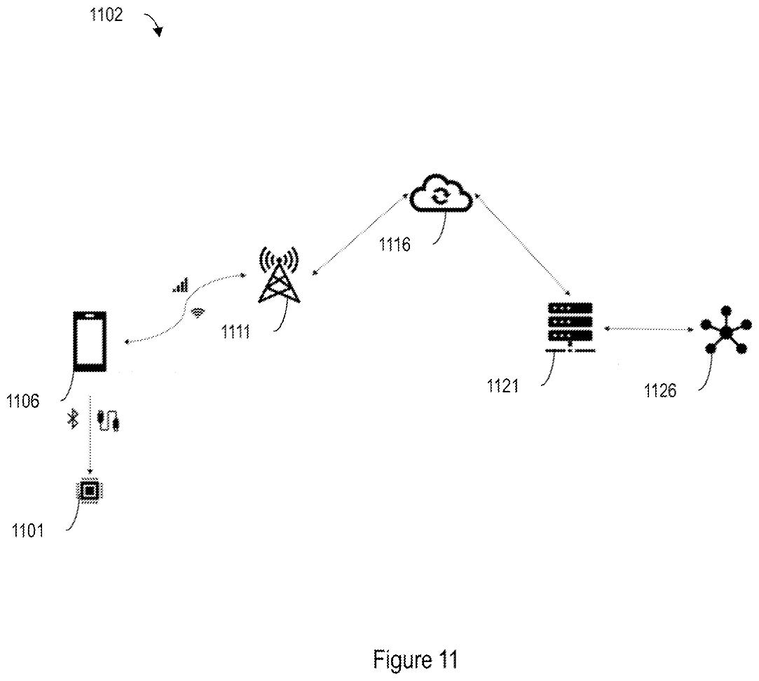

Referring to FIG. 11, illustrated herein is a diagram of a system for collecting pelvic floor rehabilitation data 1102, according to an embodiment. A pelvic floor rehabilitation device 1101 collects data from at least one sensor during pelvic floor rehabilitation therapy. The data collected by the at least one sensor may include, but is not limited to, moisture data, humidity data, force data, proximity data, temperature data, data related to the placement of scar tissue, data related to the force of dilation on the vaginal wall, the elasticity of tissue and contraction of the pelvic floor muscles, data related to resistance from the vaginal canal during dilation, pH data, photoplethysmogram (PPG) data, or any combination of data thereof.

In some embodiments, the pelvic floor rehabilitation device 1101 includes a bootloader. The bootloader supports secure boot with secure firmware update to ensure that only digitally signed firmware image may be loaded to the pelvic floor rehabilitation device 1101 and that it cannot be tampered with.

The pelvic floor rehabilitation device 1101 includes application code for sensor and input monitoring. The sensor and input monitoring application code allows for reading various sensor values and processing the sensor data.

The pelvic floor rehabilitation device 1101 also includes application code for the control functions. The control function application code allows for controlling various output functions, such as for example, lubrication release, heating, cooling, or dilation.

The pelvic floor rehabilitation device 1101 also includes application code to allow for on-device data storage.

The pelvic floor rehabilitation device 1101 includes application code to allow for communication between the pelvic floor rehabilitation device 1101 and a user device 1106. The communication application code may allow for communication with a dock-station, mobile application, or a desktop application on a user device. The pelvic floor rehabilitation device 1101 may also include application code to allow for direct communication between the pelvic floor rehabilitation device 1101 and a computing device of a healthcare professional of the user.

The data collected by the at least one sensor of the pelvic floor rehabilitation device 1101 is transmitted to a user device 1106 via a wired or wireless connection. The user device 1106 receives the data from the pelvic floor rehabilitation device 1101. The user device 1106 may allow for monitoring of the data collected by the pelvic floor rehabilitation device 1101 and control of the data, as well as control of the functions of the pelvic floor rehabilitation device 1101. The user device 1106 runs an application which may provide various features including, without limitation, allowing a user to manage the pelvic floor rehabilitation sessions provided by the pelvic floor rehabilitation device 1101, user profile management, reward systems, direct communication channels to doctors and therapists, a social network, support systems, or education, training and help systems.

The data from the user device 1106 is transmitted via an online connection 1111 to a network 1116. The network connects the user device 1106 to a server 1121 where the data is stored and possibly processed.

The server 1121 runs an application which allows for storing time series data of various parameters related to a user's health condition, device sensors, usage, and user feedback.

The data may be classified. Classification may include: private (only accessible by user), doctor (accessible by healthcare professional treating the user), regional (accessible to a local medical governing body or similar organization), research and education (accessible by a university or educating body for research), and/or business partner (accessible by a commercial business partner e.g., an insurance provider). In some cases, personal identification information may be removed before sharing. In some cases, only pooled (data from multiple users), unidentifiable data may be shared. In some cases, only analyzed data but not raw data may be shared. The authorization by the user of release of the data may be revokable at any time.

The system may include a software development kit which is shareable with different parties to enable easy integration of the system.

All software and security components of the system will comply with regulations of the governing bodies for such systems, e.g., HIPAA.

The server 1121 may also analyze data at an individual level to monitor a user's progress and suggest any changes or updates to the pelvic floor rehabilitation treatment protocol to further improve efficiency of the device use for improved benefits.

The server 1121 may also analyze collective data across a user group or several user groups classified by various parameters like demographic, age, or medical history to further improve research into pelvic health.

The server 1121 may also provide for managing user data, as well as backing up user data, and user authentication. An application programming interface (API) may also be provided for the application which runs on a user device.

The collected data may be analyzed for further research into pelvic health and its treatment. The system for collecting pelvic floor rehabilitation data 1102 may also transmit, store and control access to the data in a very secure manner both for the user privacy and for abiding by regulatory requirements.

The system for collecting pelvic floor rehabilitation data 1102 also allows for sharing of data with third parties 1126 in a secure manner to protect the privacy of the user.

The system for collecting pelvic floor rehabilitation data 1102 may classify data based on a privacy classification when determining whether it may be shared with a third party. The data may be classified as private, where the data is only accessible by the user and the server 1121 for supporting the user.

The system for collecting pelvic floor rehabilitation data 1102 may also provide for personally identifiable information (PII) removal: Any data classified other than for private and for healthcare professionals may have the PII removed before sharing of the data except when explicitly allowed by the user.

The system for collecting pelvic floor rehabilitation data 1102 may also provide that only results of processed data in various forms are shared based on the data classification without revealing the raw data.

The system for collecting pelvic floor rehabilitation data 1102 may also ensure access to the data is available only based on a defined policy and may be revoked at any point in time.

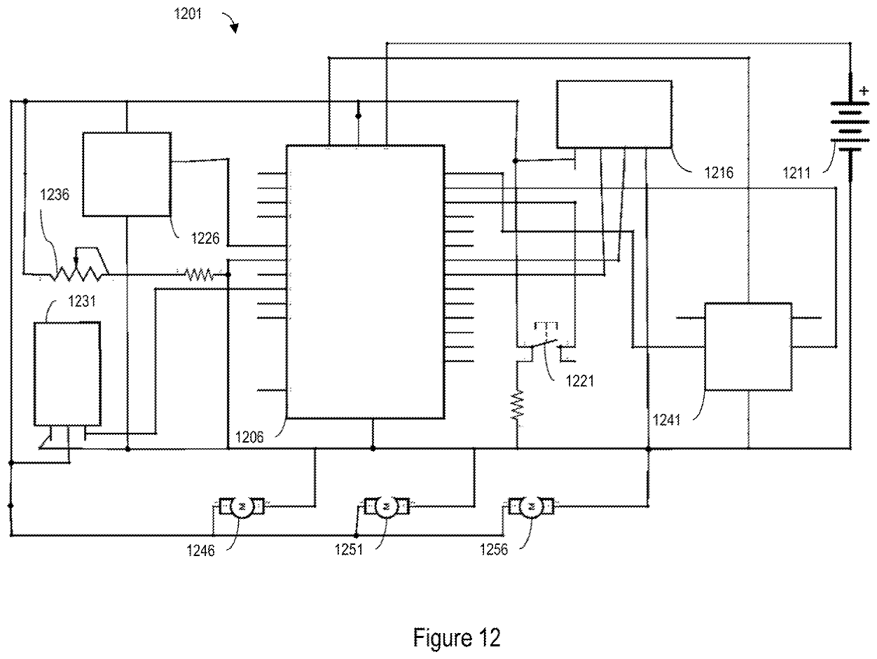

Referring to FIG. 12, illustrated herein is a circuit diagram of the circuit board of a pelvic floor rehabilitation device 1201, according to an embodiment. The pelvic floor rehabilitation device 1201 includes a microcontroller 1206 to control the operation of the components of pelvic floor rehabilitation device 1201. The microcontroller 1206 is used to provide power and logic to the sensors and electronic systems in the pelvic floor rehabilitation device 1201. The microcontroller 1206 receives data from the sensors and electronic systems in the pelvic floor rehabilitation device 1201.

The pelvic floor rehabilitation device includes a battery 1211 for powering the components of the pelvic floor rehabilitation device 1201.

The microcontroller 1206 is electronically coupled to the pressure sensor 1236 to collect data from the sensor. A switch 1221 is activated by input controls to provide power to the pressure sensor 1236 from the battery 1211. The pressure sensor 1236 allows for measuring the force of dilation on the vaginal wall, muscle, and surrounding tissue. When the pressure sensor 1236 detects pressure, the pressure sensor 1236 sends the data to the microcontroller 1206 for documentation. At the end of the session, the data is sent via the transmitter 1241 to a user device running an application for viewing the data.

The pressure sensor 1236 may also be used to measure the diameter of the dilation system 1256. When the user, the user device, or the pelvic floor rehabilitation device 1201 determines the dilation system is at an appropriate size for the pelvic floor rehabilitation session, the pressure sensor 1236 will take the measurement of the diameter to send to the microcontroller 1206 for documentation.

The pressure sensor 1236 may also work in tandem with the proximity sensor 1216 for detecting whether a particular tissue is scar tissue or is at the end of the vaginal canal.

The pressure sensor 1236 may be any sensor capable of detecting force applied in a vaginal canal which includes, without limitation, a force sensitive resistor or a flex sensor.

The microcontroller is electronically coupled to the proximity sensor 1216 to collect data from the sensor. A switch 1221 is activated by input controls to provide power to the proximity sensor 1216 from the battery 1211. The proximity sensor 1216 is used to determine placement of scar tissue and the end of the vaginal canal near the cuff or cervix. Working with the pressure sensor 1236, based on distance from devices and pressure applied on the pressure sensor 1236, the microcontroller 1206 may determine if a specific tissue is scar tissue that the pelvic floor rehabilitation device 1201 may move passed, or if the tissue is the end of the vaginal canal, which the pelvic floor rehabilitation device 1201 cannot continue pushing through without injury or pain. The data collected from the proximity sensor 1216 may be transmitted to an application running on a user device, which may inform the user on future steps to take in relation to the scar tissue. The proximity sensor may be any sensor capable of detecting a distance between the pelvic floor rehabilitation device 1201 and the vaginal cuff or cervix, which includes, without limitation an ultrasonic sensor or an infrared proximity sensor.

The microcontroller 1206 is electronically coupled to the moisture sensor 1226 to collect data from the sensor. A switch 1221 is activated by input controls to provide power to the moisture sensor 1226 from the battery 1211. The moisture sensor 1226 measures the moisture present in the vaginal canal. When the moisture sensor 1226 is turned on, the moisture sensor 1226 will measure the moisture in the vaginal canal, send the data to the microcontroller 1206, which will determine if more lubrication is needed. If more lubrication is needed the lubrication system 1251 is turned on to release lubricant into the vaginal canal. The moisture sensor 1226 may be any sensor capable of detecting moisture in a vaginal canal, which includes, without limitation, a capacitive moisture sensor, a resistive moisture sensor, or a humidity sensor.

The microcontroller 1206 is electronically coupled to the temperature sensor 1231 to collect data from the sensor. A switch 1221 is activated by input controls to provide power to the temperature sensor 1231 from the battery 1211. The temperature sensor 1231 allows for measuring the temperature inside the vaginal canal. When the temperature sensor 1231 is turned on, the temperature sensor 1231 will detect temperature and send the data to the microcontroller for documentation. At the end of the session (or during the session), the data is sent via the transmitter 1241 to a user device running an application which allows the user to see the data. The temperature sensor may measure the temperature of the temperature system 1246.

Input controls 1221 on the pelvic floor rehabilitation device 1201 provide for user input on the device to control the device functions. The input controls may include, without limitation, touch screen controls, touchpad controls, or physical buttons. The input controls 1221 allows the user to control the pelvic floor rehabilitation device 1201 at the pelvic floor rehabilitation device 1201 to alter functional parameters instead of controlling all parameters at the user device running an application.

The battery 1211 provides power for the components of the pelvic floor rehabilitation device 1201. The battery 1211 is preferably rechargeable. The battery 1211 may be located in the handle of the pelvic floor rehabilitation device 1201 and may be fixed within the device such that it cannot be removed. The battery may have an amperage between 900-1200 mAh. The battery may have a voltage between 3-6V.

While a battery 1211 is shown, it will be readily apparent that any form of a power source may be used such as a direct power supply to a wall outlet.

The temperature sensor may be used in conjunction with the control of the temperature system 1246 while generating heating or cooling. The temperature sensor 1231 thereby allows for monitoring the temperature system 1246 while the temperature system 1246 is on.

The temperature sensor 1231 may be any sensor capable of detecting temperature inside a vaginal canal which includes, without limitation, a digital temperature sensor or an analog temperature sensor.

The microcontroller 1206 is electronically coupled to the transmitter 1241 to collect data from the sensor. The microcontroller 1206 controls whether power is provided to the transmitter 1241 from the battery 1211. The transmitter 1241 may be a Bluetooth module to allow the pelvic floor rehabilitation device 1201 to wirelessly connect to a user device running an application that is compatible with the pelvic floor rehabilitation device 1201 and to send data from the pelvic floor rehabilitation device 1201 to the user device. The user device may include, without limitation, a smartphone, a laptop, a desktop, or a tablet. The transmitter allows for sending data to the application and receiving commands from the application. The transmitter may be any transmitter capable of transmitting data including, without limitation, a wired connection, a Bluetooth module, a Wi-Fi module, or a Near Field Communication (NFC) module.

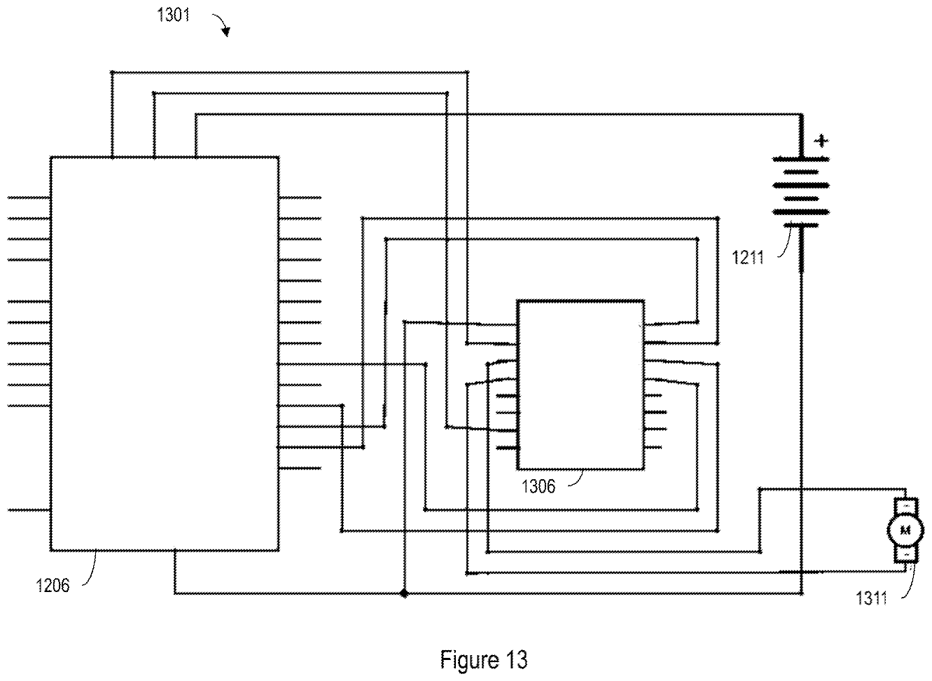

Referring to FIG. 13, a circuit board diagram is shown of the dilation system 1301 of the shaft, according to an embodiment. The dilation system 1301 of the shaft includes a motor controller 1306 for controlling the air pump 1311. The microcontroller 1206 is connected to the motor controller 1306 to allow for control of the power state of the motor controller 1306. The motor controller 1306 controls the power to the air pump 1311 and controls the rate by which the air pump 1311 pumps air into the dilation elements. The power for the dilation system 1301 is provided by the battery 1211.

The lubrication system and the temperature adjusting system may have similar circuit board configurations to the dilation system 1301.

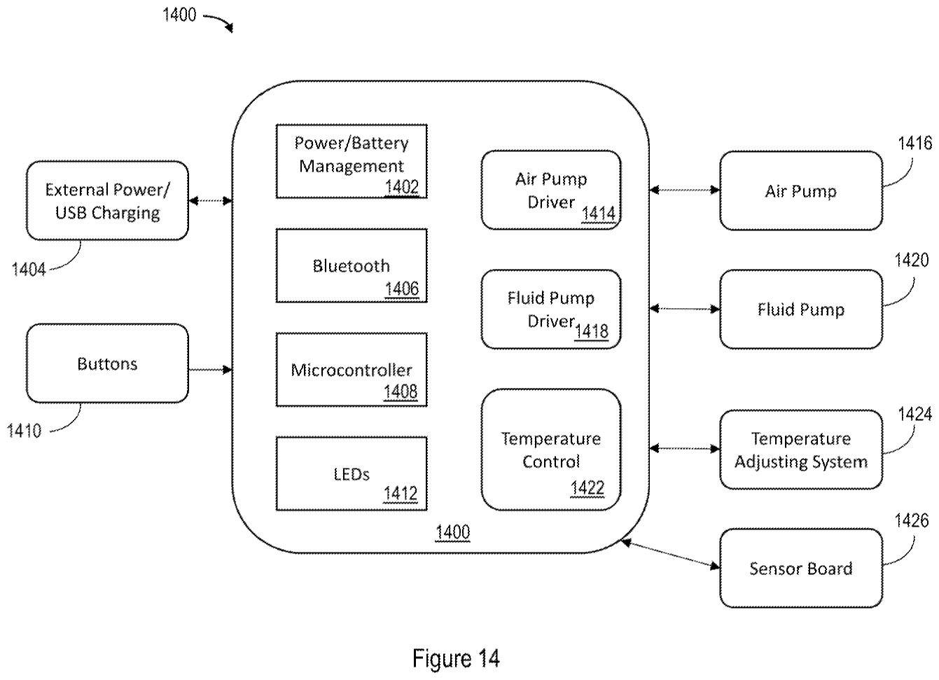

FIG. 14 is a block diagram of an electronic system 1400 of a pelvic floor rehabilitation device. The pelvic floor rehabilitation device includes a dilation system, a lubrication system, and a temperature system. In other embodiments, the pelvic floor rehabilitation device may only include a dilation system or may include a dilation system and a lubrication system, or a dilation system and a temperature system.

FIG. 14 shows an exemplary electronics configuration including a main circuit board or mainboard 1400. Power/battery management 1402 is on the mainboard and is electrically coupled to the external power and/or USB charging components of the device. The pelvic floor rehabilitation device may receive electricity directly to power the device as well as receiving power which charges a battery which then powers the device. The plug components for power are preferably located on the handle portion of the pelvic floor rehabilitation device.

The mainboard also includes Bluetooth components 1406 which allow for wireless communication with external computing devices (e.g., mobile phone or tablet).

The mainboard includes a microcontroller which outputs commands to control various components of the device as well as receiving inputs from various components of the device including sensors and external buttons 1410. The buttons 1410 can be pressed by the user to perform various functions, for example turning the device off or on, starting a session, providing lubrication, decreasing dilation, etc.

The mainboard includes at least one light-emitting diode (LED) 1412 which indicates a state of the device to the user. For example, an LED may show that the device is on or off, that the device needs charging, or that the device has an error.

The mainboard includes air pump driver 1414 which is electrically coupled to the air pump 1416 to turn the air pump on or off, and in some embodiments to regulate the rate of flow of air being pumped by the air pump 1416.

The mainboard includes fluid pump driver 1418 which is electrically coupled to fluid pump 1420 to turn the fluid pump 1420 on or off, and in some embodiments to regulate the rate of flow of fluid (lubrication and/or medication) being pumped by the fluid pump 1420.

The mainboard includes a temperature control 1422 which is electrically coupled to the temperature adjusting system 1424 to turn the temperature adjusting elements off or on, and in some embodiments to control a rate at which the temperature is increased or decreased.

The electronic system 1400 further includes a sensor board 1426 which is coupled to at least one sensor within the device to receive information regarding at least one parameter of the operation of the device or of the vagina of the user and transmit the data to the microcontroller at the mainboard to either augment the operation of the device or to eventually provide data to the user and/or a healthcare professional.

In some embodiments the electronic system 1400 may include other system controls which connect to other systems as described above. The other system may include an EMG system, a vibration system, a cardiovascular sensor system, and/or a camera system.

FIG. 14 represents one embodiment of electrical and electronic connections between the components of the pelvic floor rehabilitation device. In other embodiments the various components shown on the mainboard may be located elsewhere within the device or on another board. There may be more than two boards (mainboard and sensor board) or only one board. The pelvic floor device may include only the air pump system, just the air pump system and the fluid system, or just the air pump system and the temperature adjusting system. Other systems may also be present. Bluetooth connectivity may not be present. Data may be stored locally on the device before being transferred wirelessly (but not via Bluetooth) or via wire to another device for analysis and reporting.

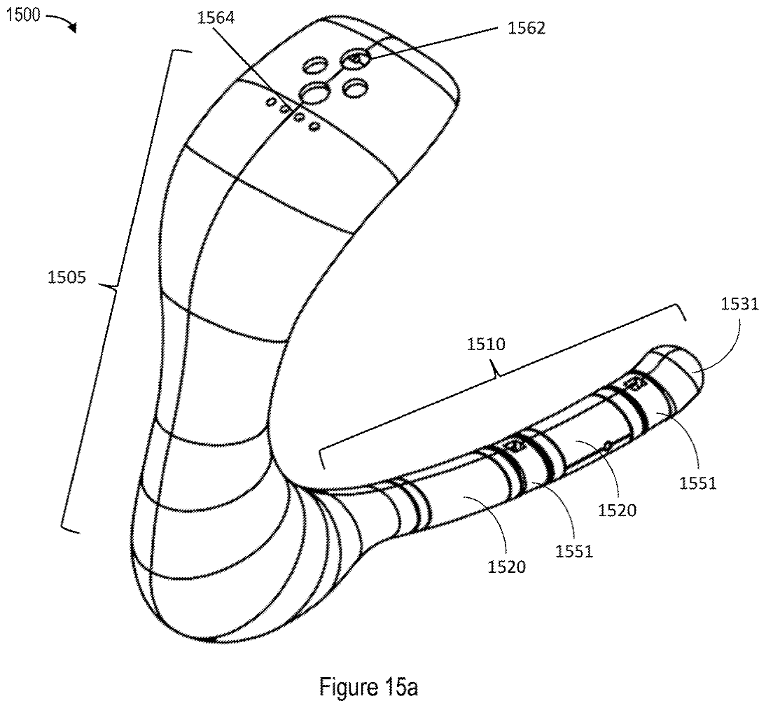

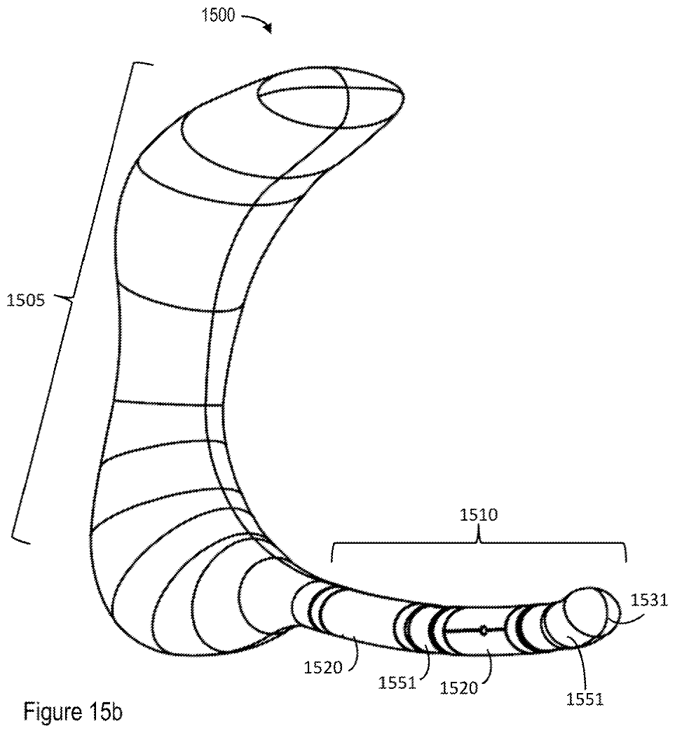

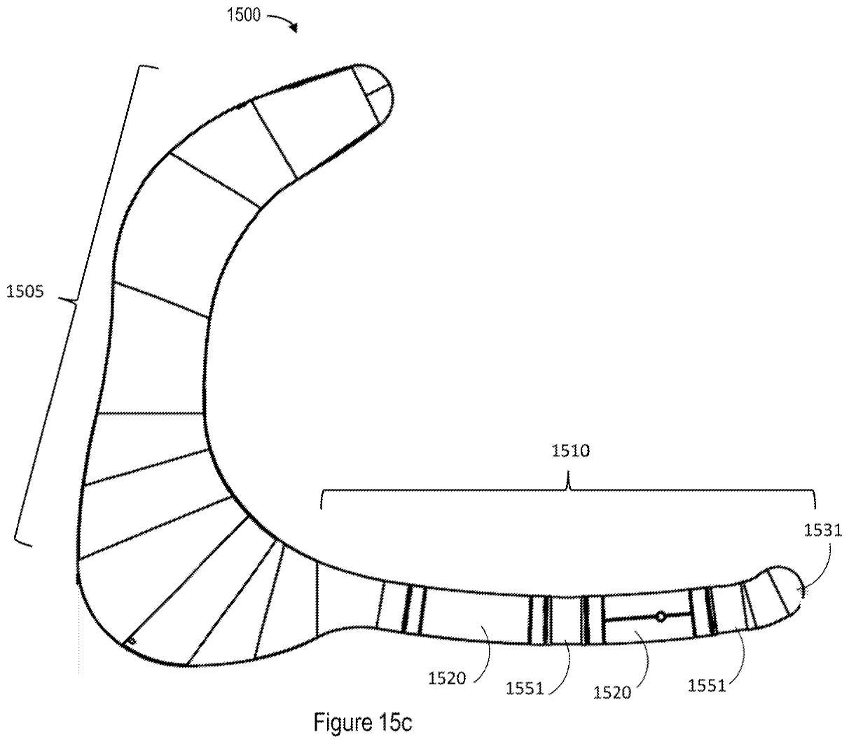

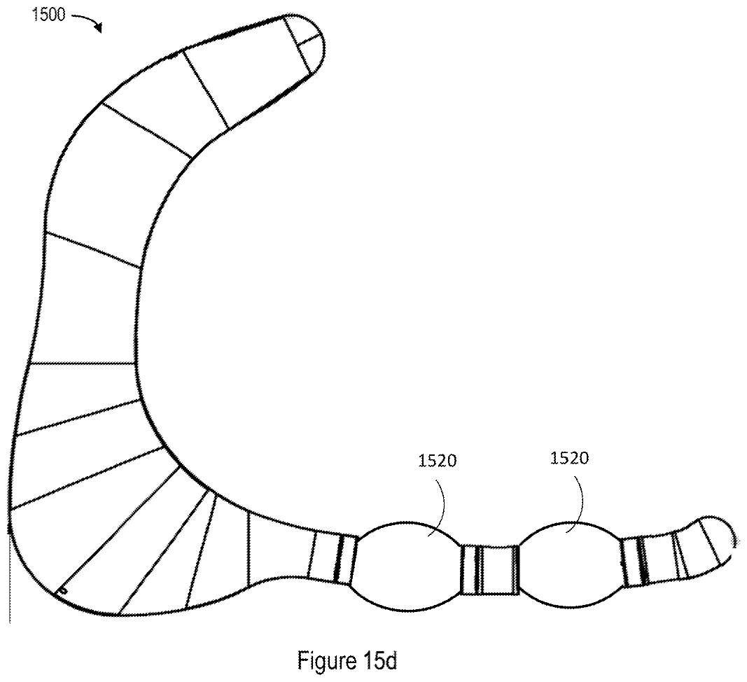

FIG. 15a is a perspective view of a pelvic floor rehabilitation device according to an embodiment.

Referring now to FIG. 15a-d, illustrated therein are various perspective views of a pelvic floor rehabilitation device 1500, according to an embodiment. The embodiment of FIG. 15a-d is an ergonomic design that could be employed by a user as compared to the simplified design shown in earlier FIGS. 5-10.

In FIG. 15a a first perspective view is shown. The pelvic floor rehabilitation device 1500 includes a handle 1505 and a shaft 1510. The shaft 1510 is inserted into the vagina by a user. In use, the user can hold onto the handle 1505 while inserting the shaft 1510 into the vagina and while a treatment session is occurring.

The handle 1505 includes four buttons 1562 (larger circles, only one labelled to reduce clutter) by the use of which the user can control the operation of the pelvic floor rehabilitation device 1500. For example, the buttons may turn the pelvic floor rehabilitation device 1500 on or off, connect the pelvic floor rehabilitation device 1500 to a computing device (e.g. the user's mobile device), and/or allow the user to adjust parameters during operation of the pelvic floor rehabilitation device 1500 (e.g. decrease dilation, increase lubrication, decrease temperature, etc.). In other embodiments the button may be located anywhere which is accessible by the user during use, for example, on the underside of the handle (opposite the location in FIG. 15a).

The handle 1505 includes four light emitting diodes (LEDs) 1564 (smaller circles, only one labelled to reduce clutter) which may signal to the user various information about the pelvic floor rehabilitation device 1500. For example, one or more LEDs may light up to show the user that the pelvic floor rehabilitation device 1500 is connected to a user mobile device (e.g. a Bluetooth connection has been established), one or more LEDs may show the user that the pelvic floor rehabilitation device 1500 is charged or requires charging, and/or one or more LEDs may show the user their progress during a session of use of the pelvic floor rehabilitation device 1500.

The shaft 1510 include two dilation elements 1520, two temperature adjusting elements 1551, and an end 1531.

In operation, one or both of the dilation elements 1520 may dilate (both are capable of dilation). The dilation may be achieved by a pneumatic air pump system which injects air into an interior of the dilation elements 1520. The dilation elements do not inflate uniformly and are larger in the middle than at either end when inflated. The dilation elements 1520 may have a maximum diameter of dilation of 41 mm at the largest part. The dilation elements 1520 may have a minimum diameter of 18-20 mm (smallest point-largest point).

The dilation elements 1520 may inflate under the control of an electrical deflation system which employs two solenoid valves wherein when the solenoids are powered on by the electrical system a port at a first solenoid valve is open allowing air to flow through into the dilation elements 1520 and when power is off a port at the second solenoid valve is open to allow for the air to flow out of the dilation elements 1520 to allow for deflation. In other embodiments there may be only one solenoid valve which is open when powered on and closed when powered off to control the flow of air into the dilations elements 1520 wherein deflation is achieved by other means. In some embodiments each dilation elements 1520 may have a separate solenoid valve system.

The temperature adjusting elements 1551 of the embodiment of FIGS. 15a-d are resistive heating elements. A current is applied to the resistive heating elements to heat the resistive heating elements. In the embodiment of FIGS. 15a-d cooling may be achieved by inserting a cooling rod into the shaft wherein the resistive heating elements conduct cooling to the vagina. In operation, one or both of the temperature adjusting element 1551 may increase or decrease in temperature to heat or cool the vagina. Applying heat to the vaginal walls may improve the results of a treatment session by relaxing the muscles of the walls to allow for a greater effect of dilation for the user. Cooling the vaginal walls may aid in recovery following a treatment session.

FIG. 15b is a second perspective view of the pelvic floor rehabilitation device 1500 is shown. FIG. 15b also shows the handle 1505, the shaft 1510, the dilation elements 1520, the temperature adjusting elements 1551, and the end 1531.

FIG. 15c is a third perspective view from the side of the pelvic floor rehabilitation device 1500 is shown. FIG. 15b also shows the handle 1505, the shaft 1510, the dilation elements 1520, the temperature adjusting elements 1551, and the end 1531.

FIG. 15d is the fourth perspective view of the pelvic floor rehabilitation device 1500 from the same angle as the view of FIG. 15c. In this view, however, the dilation elements 1520 have been dilated.

In the embodiment of FIG. 15a-d, the device comprises two halves of a shell which are joined along the line that runs down the back of the handle as seen in FIG. 15a. The two halves may be joined by screws, by ultrasonic welding, by glue, or by any other appropriate means of joining.

In other embodiments the pelvic floor rehabilitation device may be modular wherein the shaft portion and the handle portion can be separated. In those embodiments, the device would require a locking mechanism at the connection between the handle and the shaft. The locking mechanism would require ports which allow for tubing, wires, and other components to pass through the locking mechanism when it is in a locked position. Tubing that may need to pass through the locking mechanism may include tubing for introducing lubrication/medication into the vagina, and tubing for introducing air (or another fluid) into the dilation elements. In those embodiments, the handle would hold most of the electronic elements of the device including the battery, the circuit boards, Bluetooth components, except for the electronic components of the elements which directly interact with the user which would be within the shaft (e.g. temperature adjusting elements, pressure sensors, temperature sensors, etc.).

In embodiments where the handle and shaft can be separated there may be multiple iterations of the shaft which include different systems. For example, the shaft could include just a dilation system, just a heating system, just a cooling system, both dilation and lubrication, both dilation and heating, etc.

In embodiments where the handle and shaft can be separated, a cooling system of the pelvic floor rehabilitation device may include inserted cooling rods into the shaft to apply cooling to the vagina.

The modularity of the handle and shaft may also allow for replacement shafts to be used with a single handle in that a component of the shaft breaks and needs to be replaced. This would minimize costs as the entire device would not need to be replaced.

In the embodiments of FIG. 15a-15d, various sensors may be present at the dilation elements 1520 (i.e. pressure sensors) and the temperature adjusting elements 1551 (i.e. temperature sensors). These sensors need to be flush with a surface of the pelvic floor rehabilitation device and must not interfere with the operation of the respective elements. The sensors must also only require that wires attach to a single side of the sensor as wires cannot be outside of an interior of the pelvic floor rehabilitation device.

In some embodiments, EMG sensors (surface electrodes) may be present in the dilation elements.

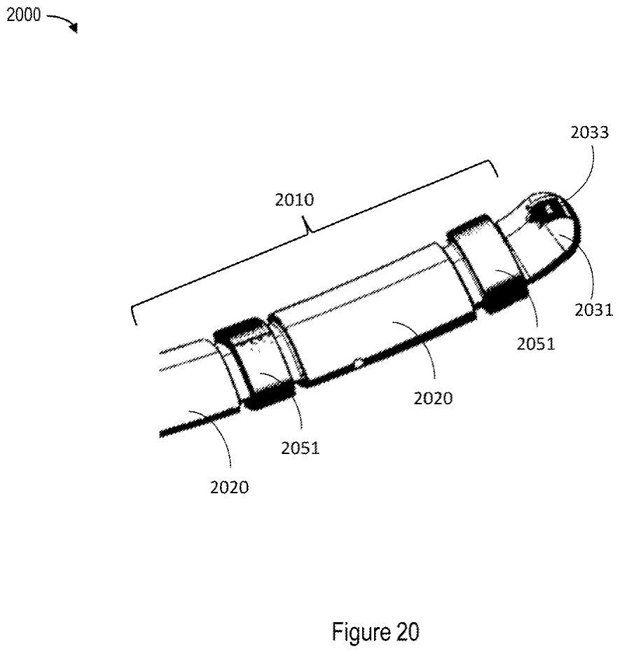

In some embodiments, other sensors may be present at the end 1531 of the pelvic floor rehabilitation device. The sensors at the end 1531 may be on a single sensor board, multiple sensor boards, or may be individual sensors. The sensors at the tip may include, as discussed above: moisture sensors, pH sensors, force sensors, blood oxygen sensors, heart rate sensors, oxygen saturation sensors, pulse oximeters, radiation sensors, ultrasound, accelerometers, and proximity sensors may be present at either the dilation elements or the temperature adjusting elements or at the end 1531. FIG. 20, discussed below, shows an embodiment with a sensor board at the tip.

A camera may be present at end 1531.

A vibration system may be present within the shaft 1510.

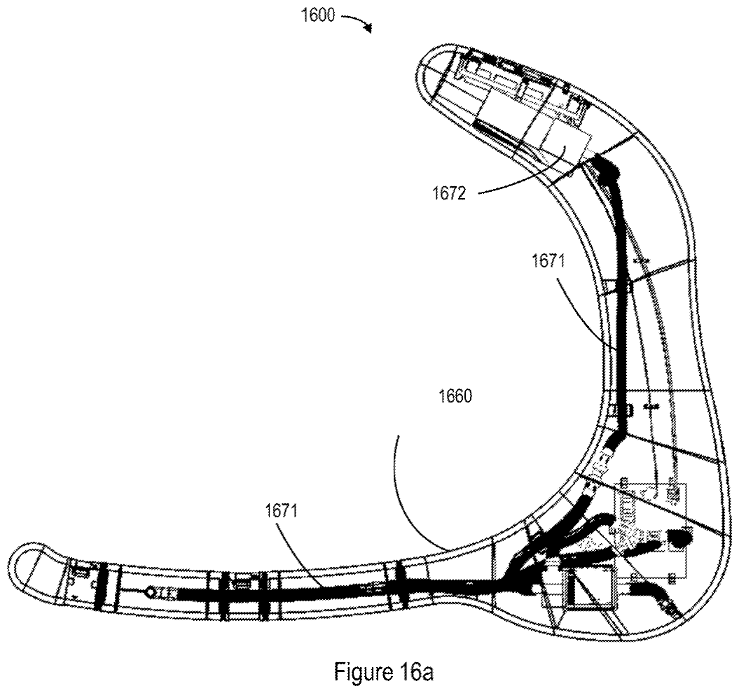

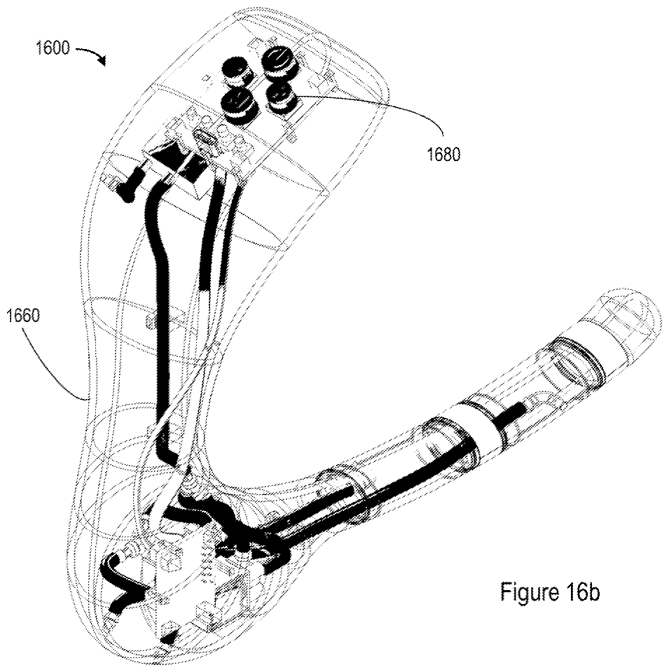

FIGS. 16a and 16b are transparent perspective views of a pelvic floor rehabilitation device 1600 including a dilation system, according to an embodiment.

FIG. 16a shows the same pelvic floor rehabilitation device 1600 as the pelvic floor rehabilitation device 1500 of FIGS. 15a-d. In FIG. 16, the device is shown as transparent and components of a dilation system which functions to inflate the dilation elements are seen.

FIG. 16a is an example of how the components of a system of the pelvic floor rehabilitation device 1600 (e.g. a dilation system, a lubrication system, a temperature system) fit inside the exterior shell 1660 of the device 1600. Only some elements of the dilation system have been shown, and only some elements have been called out. Shown is tubing 1671 through which air passes to inflate the dilation segments of the pelvic floor rehabilitation device, as well as an air pump 1672 which pumps air into the tubing 1671. Shown but not labelled are additional elements of the dilation system including electronic elements which power the pump and various valves, and connect the system to a controller of the device.

FIG. 16b is a different perspective view of the same dilation system as FIG. 16a within the shell 1660 of the pelvic floor rehabilitation system 1600. In this view the buttons 1680 which a user can use to control the pelvic floor rehabilitation device 1600, including the dilation system, can be seen.

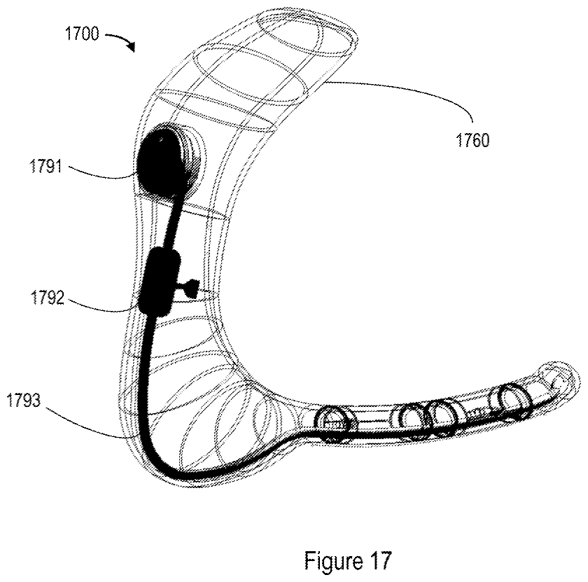

FIG. 17 is a transparent perspective view of a pelvic floor rehabilitation device including a lubrication system, according to an embodiment.

FIG. 17 shows the same pelvic floor rehabilitation device 1700 as the pelvic floor rehabilitation device 1500 of FIGS. 15a-d. In FIG. 17, the device is shown as transparent and components of a lubrication system which functions to lubricate the vagina are seen. These components are only examples and are shown to illustrate how the various systems of the pelvic floor rehabilitation device fit inside the pelvic floor rehabilitation device. The lubrication system includes a lubrication cartridge 1791 which is placed in the handle portion of the pelvic floor rehabilitation device 1700, a lubrication pump 1792, and tubing 1793 through which the lubricant is delivered to the vagina through an opening at the tip of the device 1700.

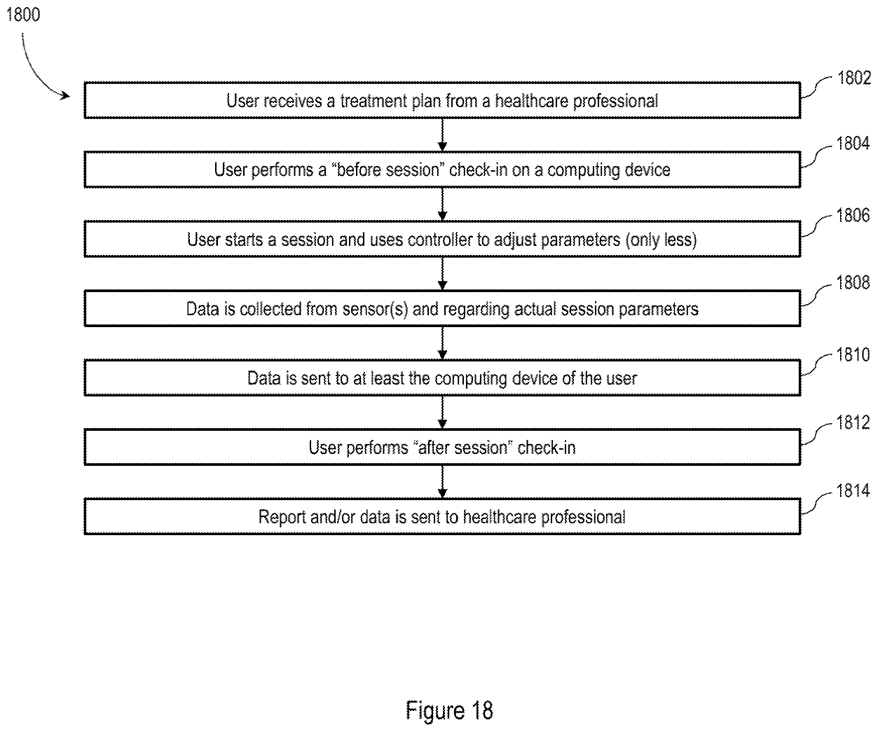

FIG. 18 is a flow diagram of a method 1800 of using a pelvic floor rehabilitation device system, according to an embodiment. The method 1800 is for a collaboration between a patient (the user) and at least one healthcare professional. The patient is prescribed a pelvic floor rehabilitation device by their healthcare professional and works together with the healthcare professional to establish a treatment plan to follow.

At 1802, the user receives a treatment plan from their healthcare professional. Preferably, the treatment plan is automatically loaded into an application which controls the pelvic floor rehabilitation device by the healthcare professional. That is the healthcare professional creates a treatment plan which is loaded into a pelvic floor rehabilitation software application which is then accessed by the user either on a computing device or by the pelvic floor rehabilitation device itself to load the treatment plan parameters into the device. For example, the user may log in to a pelvic floor 47 rehabilitation mobile application on their mobile phone to access a preprogrammed treatment plan and then connect the device to their mobile phone to load the treatment plan parameters into the device. In some embodiments, the user may receive the treatment plan and be required to input the parameters of the treatment plan into an application on their computing device.

At 1804, the user logs in to the pelvic floor rehabilitation application on their computing device (e.g. mobile phone, tablet, laptop) and performs a “before session” check-in. This may include answering various questions about their general health or specific pelvic floor rehabilitation health, questions regarding how they feel following past treatments, questions about physical activity or sexual activity they have undertaken, etc.

At 1806, the user starts the treatment plan session. The user may insert the pelvic floor rehabilitation device into their vagina and then start the session or they may start the session and sensors on the pelvic floor rehabilitation device may output information which informs the device when to start various aspects of the session, for example dilation of dilations elements. During the session the user may adjust the parameters of the treatment plan but only to decrease the intensity of a parameter. For example, the user can only decrease the amount of inflation that has been prescribed or can only decrease the amount of heating that has been prescribed. Adjustment may be done at buttons on the handle of the device or on a computing device which is running the pelvic floor rehabilitation application.

At 1808, during the session, data is collected by sensors of the pelvic floor rehabilitation device (e.g. pressure sensors, temperature sensors, moisture sensors, pH sensors, PPG/VPP sensors, etc.) and directly from the systems of the pelvic floor rehabilitation device (e.g. dilation system, lubrication system, temperature system, etc.). The data collected provides a full picture of the functioning of the system (e.g. did the heating system heat properly), the effects of the pelvic floor rehabilitation device (e.g., is their less pressure from the vaginal wall then in previous sessions), and the actual parameters used (e.g. did the user decrease the dilation from the prescribed dilation).

At 1810, the data is sent from the pelvic floor rehabilitation device to at least a computing device of the user (e.g. a mobile phone, laptop, or tablet). The data may be sent via Bluetooth to the computing device. In some embodiments, there may be a wired connection between the pelvic floor rehabilitation device and the computing device (e.g. a USB-C connection). In some embodiments, the data may be sent directly to a computing device of a healthcare professional (e.g. if the session is occurring at a healthcare office).

At 1812, the user performs an “after session” check-in. The user may answer questions about the session to provide additional information which may be important. For example, the user may be prompted to explain why they deviated from the prescribed treatment plan, and/or the user may be asked to answer questions about how they felt during the session and what effects they think occurred due to the session.

At 1814, a report and/or data is sent to the healthcare professional of the user. The data collected from the before and after session check-ins as well as the session itself may be collected together and sent as raw data or in the form of a report. The data may be analyzed to create refined data or to create a report. The data collected and sent and/or the report may be used by the healthcare professional and the user to make decisions about future treatment sessions. The data and/or report may be flagged or include flags which alert the user and/or the healthcare professional of issues such as unexpected results or errors which occurred during the session.

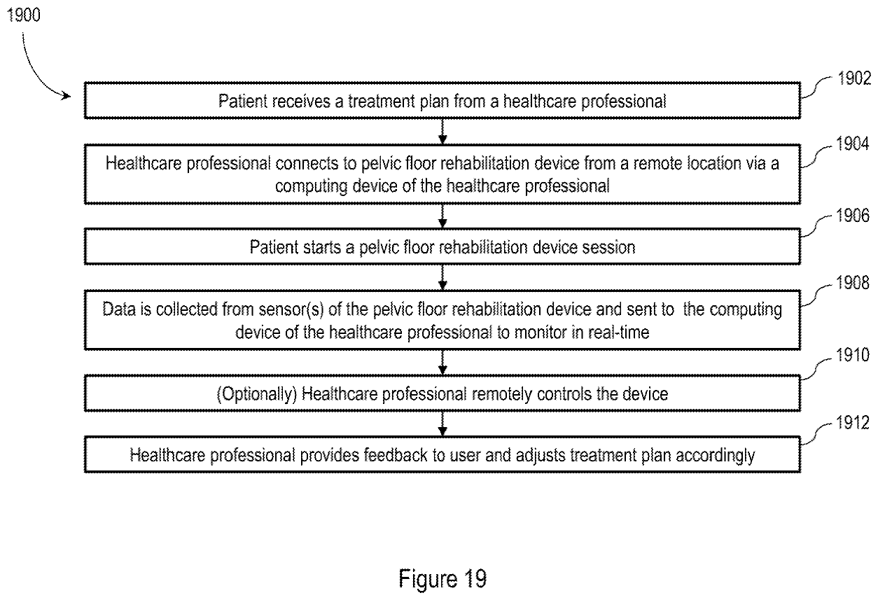

FIG. 19 is a flow diagram of a method of remote therapeutic monitoring of use of a pelvic floor rehabilitation device in accordance with the embodiments described herein. Remote therapeutic monitoring removes barriers to treatment because the patient and the healthcare professional do not need to be in the same room, the patient does not need to travel, space does not need to be taken within a healthcare setting, and the patient can be in an environment they find comfortable. Remote therapeutic monitoring saves time for both the patient and healthcare professionals.

Remote therapeutic monitoring allows the healthcare professional(s) to understand changes happening to the patient as they receive treatment from home (or wherever they choose) and to ensure that the patient following the treatment plan, is improving as expected, and does not need to see the doctor in person.

At 1902, a patient/user receives treatment plan from a healthcare professional (e.g., a medical doctor).

At 1904, the healthcare professional connects remotely to a pelvic floor rehabilitation device of the patient through a computing device of the healthcare professional such that the computing device of the healthcare professional is communicatively coupled to the pelvic floor rehabilitation device and configured to receive sensor data from a plurality of sensors of the pelvic floor rehabilitation device.

At 1906, the patient starts a pelvic floor rehabilitation device session which follows the treatment plan as provided to the patient by the healthcare professional.

At 1908, while the session is occurring, data is collected from the plurality of sensors of the pelvic floor rehabilitation device and sent to the computing device of the healthcare professional in real-time so that the healthcare professional can monitor the treatment session. The healthcare professional views the sensor data on a graphical user interface (GUI) of the computing device of the healthcare professional. The computing device may include an input device for the healthcare professional to provide input to the computing device of the healthcare professional as well as the pelvic floor rehabilitation device. The sensor data shown to the healthcare professional on the GUI may include raw data and/or processed data.

The computing device of the healthcare professional may also receive patient-reported data generated by the patient during the session. This further enables the healthcare professional and the patient to create a feedback loop between them which allows for real-time communication and monitoring of the device.

The sensor data may be received at a computing device of the patient and then sent to the computing device of the healthcare professional or may be sent directly from the pelvic floor rehabilitation device to the computing device of the healthcare professional. The sensor data and patient-reported data may be sent through a cloud server. The sensor data and patient-reported data may be stored on a cloud server. The sensor data and patient-reported data may be sent to the computing device of the healthcare professional through a Bluetooth™ connection or a 5G network. That is, the data can flow from the patient to the healthcare professional is any suitable and safe manner.

In FIG. 19, the healthcare professional is at a remote location physically removed from the patient, however, in other embodiments the healthcare professional may be physically near the patient but still connected to the pelvic floor rehabilitation device only remotely through the computing device of the healthcare professional.

At 1910, in an optional step, the healthcare professional may remotely control the device to alter the treatment plan. For example, the healthcare professional may notice that a desired result is not occurring and may remotely manually control the device or remotely add or remove steps or increase or decrease parameters, or may remotely stop the session entirely.

At 1912, during the session and/or after the session is over, the healthcare professional provides feedback to the user about the session and may adjust the treatment plan as they determine is necessary based on the sensor data received during the session. The healthcare professional may view data analytics of the treatment session based on the sensor data and compare to previous patient data or data from other patients when providing feedback or changing treatment plans.

Analysis of the data collected during a session may provide baseline measurements for a treatment plan and progress tracking of the treatment plan, as well as future baseline measurements for treatment and/or diagnosis of pelvic floor and gynecological conditions not currently being treated.

The remote monitoring system and method of FIG. 19 may be applied in cases of tissue healing, muscle control and response, function of surrounding organs, metabolic conditions, nervous system response, etc.

The remote monitoring system and data collected may be used to measure, monitor, analyze, compare, and diagnose symptoms and conditions active within the pelvic floor, vaginal vault and surrounding pelvic structures.

FIG. 20 shows an embodiment of a pelvic floor rehabilitation device 2000 with a sensor board 2033 at an end 2031 which includes multiple different sensors.

FIG. 20 is a view of only the shaft 2010 of the pelvic floor rehabilitation device 2000. The shaft includes dilation elements 2020 and temperature elements 2051 similar to dilation elements 1520 and temperature elements 1551 as shown and described in FIG. 15a-d.

The end 2031 of the shaft 2010 includes a sensor board 2033 which includes several sensors. The sensors may include at least one of, as described above, an oxygen saturation (SPO₂), heart rate sensor, blood oxygen sensor, pulse oximeter, radiation sensor, ultrasound sensor, pH sensor, moisture sensor, force sensor, proximity sensor, accelerometer, and temperature sensor.

Multiple embodiments have been described herein. It is to be understood that in some embodiment the pelvic floor rehabilitation device may only include a dilation system, while in other embodiments the device may include a dilation system and a lubrication system, or a dilation system and a temperature system. Other systems may be included in the device.

Herein, multiple different types of sensors have been discussed. It is to be understood that any number of each type of sensor may be used in the device. For example, the device may include three temperature sensors or two pressure sensors.

While the above description provides examples of one or more apparatus, methods, or systems, it will be appreciated that other apparatus, methods, or systems may be within the scope of the claims as interpreted by one of skill in the art.

1-70. (canceled)

71. A pelvic floor rehabilitation device comprising:
a handle for holding and manipulating the pelvic floor rehabilitation device;
a controller to receive instructions and to control operation of the pelvic floor rehabilitation device;
a shaft extending from the handle, wherein at least part of the shaft dilates based on input from the controller;
at least one sensor for collecting data during operation of the pelvic floor rehabilitation device; and
a lubrication system operable to introduce a substance into the vagina.

72. The pelvic floor rehabilitation device of claim 71 wherein the substance introduced by the lubrication system includes at least a pharmaceutical.

73. The pelvic floor rehabilitation device of claim 72 wherein the pharmaceutical is contained within a lubrication cartridge.

74. The pelvic floor rehabilitation device of claim 73 wherein the lubrication cartridge contains at least one of a lubricant and a fluid.

75. The pelvic floor rehabilitation device of claim 72 wherein the at least a pharmaceutical is personalized for a patient.

76. The pelvic floor rehabilitation device of claim 72 wherein the pharmaceutical is used for at least one of desensitization, pain relief, and hormone replacement.

77. The pelvic floor rehabilitation device of claim 72 wherein the pharmaceutical is at least one of an estrogen, a progesterone, lidocaine, morphine, valium, a muscle relaxant, cannabidiol (CBD), and tetrahydrocannabinol (THC).

78. The pelvic floor rehabilitation device of claim 73 wherein the lubrication system includes an identification system for identifying the type of lubrication cartridge inserted into the lubrication system.

79. The pelvic floor rehabilitation device of claim 78 wherein the identification system includes a barcode scanner to scan a barcode on the lubrication cartridge to identify the lubrication cartridge.

80. The pelvic floor rehabilitation device of claim 73 wherein the lubrication system may include a puncturing mechanism for puncturing the lubrication cartridge when the lubrication cartridge is inserted into the lubrication system, wherein a puncture hole produced by the needle allows for drawing the contents from the lubrication cartridge by the lubrication system.

81. The pelvic floor rehabilitation device of claim 71 further comprising a transmitter for transmitting data collected by the at least one sensor to at least a first computing device.

82. The pelvic floor rehabilitation device of claim 71 wherein the controller is communicatively coupled to a computing device which transmits instructions for operation of the pelvic floor rehabilitation device.

83. The pelvic floor rehabilitation device of claim 71 wherein the handle includes at least one input control engageable by a user and communicatively coupled to the controller to control operation of the pelvic floor rehabilitation device.

84. The pelvic floor rehabilitation device of claim 71 further comprising a temperature adjusting system operable to alter a temperature in a vagina.

85. The pelvic floor rehabilitation device of claim 71 wherein the at least one sensor comprises a pressure sensor to detect a resistance from a vagina.

86. The pelvic floor rehabilitation device of claim 71 wherein the at least one sensor comprises a force sensor to measure the force of dilation on a vagina.

87. The pelvic floor rehabilitation device of claim 71 wherein the at least one sensor comprises a temperature sensor to measure the temperature inside a vagina.

88. The pelvic floor rehabilitation device of claim 71 wherein the at least one sensor comprises a moisture sensor to measure a level of moisture inside a vagina.

89. The pelvic floor rehabilitation device of claim 71 further comprising an electromyography (EMG) system for tracking and measuring pelvic floor dysfunction using surface electrodes on a surface of the shaft.

90. The pelvic floor rehabilitation device of claim 71 further comprising an ultrasound system for elastography and imaging.
